(19) United States
(12) Patent Application Publication
(10) Pub. No.: US 2014/0004539 A1
(43) Pub. Date: Jan. 2, 2014

(54) SYSTEMS AND METHODS FOR MULTIPLEX SOLUTION ASSAYS

(71) Applicants: Arlyne Simon, Ann Arbor, MI (US); John Frampton, Ann Arbor, MI (US); Josh White, Ann Arbor, MI (US); Shuichi Takayama, Ann Arbor, MI (US)

(72) Inventors: Arlyne Simon, Ann Arbor, MI (US); John Frampton, Ann Arbor, MI (US); Josh White, Ann Arbor, MI (US); Shuichi Takayama, Ann Arbor, MI (US)

(73) Assignee: THE REGENTS OF THE UNIVERSITY OF MICHIGAN, Ann Arbor, MI (US)

(21) Appl. No.: 13/918,494

(22) Filed: Jun. 14, 2013

Related U.S. Application Data
(60) Provisional application No. 61/660,249, filed on Jun. 15, 2012.

(51) Int. Cl. G01N 33/53

(52) U.S. Cl. CPC G01N 33/5306
USPC 435/7.92; 436/501; 435/7.1

(57) ABSTRACT

The present invention relates to systems and methods for an aqueous 2-phase system droplet array and uses thereof. The present invention further relates to methods for performing multiplexed assays, chemical, and/or biological experiments and tests (e.g., immunoassays).

This application claims priority to U.S. Provisional Application No. 61/660,249, filed Jun. 15, 2012, which is herein incorporated by reference in its entirety.

### FIELD OF THE INVENTION

The present invention relates to systems and methods for an aqueous 2-phase system droplet array and uses thereof. The present invention further relates to methods for performing multiplexed assays, chemical, and/or biological experiments and tests (e.g., immunoassays).

### BACKGROUND OF THE INVENTION

The fields of life science research and pharmaceutical development are dependent upon highly selective and sensitive quantitative assays for a wide range of different biomolecules (such as proteins, antibodies, cytokines, receptors, enzymes, peptides, nucleic acids, hormones, and the like) in complex clinical or biological samples (such as blood, urine, tissue or cellular extracts, cell culture supernatants, bioprocess feedstreams, and the like). In typical samples (which may contain thousands of different molecular species) the analytes of interest may be present at extremely low concentrations (nanograms per milliliter or less), but the samples may be available only in very small quantities (microliters or less). The rapid growth in the field of biotechnology and the introduction of many potential new drug targets from genomic research have created an increasing demand for more rapid and efficient analytical methods, without any sacrifice in performance.

In order to simultaneously obtain high selectivity (the ability to measure one very specific molecule in a complex mixture) and high sensitivity (the ability to accurately quantify very small concentrations or amounts), a number of analytical methods have been developed which couple powerful molecular separations with extremely responsive detection methods.

Existing methods, such as ELISA assays suffer from insufficient sensitivity and specificity and are expensive. New methods are needed for diagnostic assays to allow for sensitive and specific detection of analytes.

### SUMMARY OF THE INVENTION

The present invention relates to systems and methods for an aqueous 2-phase system droplet array and uses thereof. The present invention further relates to methods for performing multiplexed assays, chemical, and/or biological experiments and tests (e.g., immunoassays).

For example, embodiments of the present invention provide supports, systems, kits, and methods for performing multiplexed assays (e.g., immunoassays), comprising: a) a solid support comprising at least one first defined area (e.g., topographically or chemically defined); and optionally two or more second defined areas within the first defined area; and b) a first solution comprising a first polymer or solute (e.g., salt) and reagents for detecting the presence or absence of one or more analytes in a test sample; and c) a second solution comprising a second polymer or solute (e.g., salt) and the test sample, wherein the second solution has a different density than the first solution, and wherein the first and second solutions form an aqueous two-phase system when mixed. In some embodiments, the first and second topographically defined areas are selected from, for example, wells, hydrophobic lines, and physical barriers. In some embodiments, the solid support is, for example, a plate, a multiwall plate, or a slide. In some embodiments, the solid support is constructed from a material selected from, for example, plastic, paper, hydrogel, tape, cloth, yarn or thread. In some embodiments, the reagents comprise an antibody specific for the analyte. In some embodiments, the test sample is, for example, plasma, serum, urine, saliva, body fluids, cell culture media, or cells. In some embodiments, the solid support is a 6, 24, 48, 96, 384, 1536, 3456 or 9600 well microplate. In some embodiments, the first and second polymers are selected from, for example, polyethylene glycol (PEG), dextran (DEX), DEX-methylcellulose, DEX-polyvinyl alcohol, PEG-DEX sulfate, polyvinyl alcohol-DEX sulfate, hydroxypropyldextran-DEX, DEX sulfate-methylcellulose, or ethylene oxide-propylene oxide (EOPO). In some embodiments, the first or second solutions comprise two or more polymers. In some embodiments, the reagents comprise one or more of primary and secondary antibodies that specifically binds to analytes in the test sample. In some embodiments, the antibodies are attached to the surface of the solid support. In some embodiments, one or more of the first solution and the second solution is dehydrated (e.g., rehydrated upon addition of the first or second polymer, test sample, water, or other aqueous liquid). In some embodiments, multiple droplets of the first and/or second solutions contain different concentrations of capture and detection antibodies against the same antigen. In some embodiments, the first and/or second solutions comprise one or more reagent selected from, for example, primary antibodies, secondary antibodies, antibody-conjugated acceptor beads, biotinylated detection antibodies, enzymes, reagents for amplification reactions, probes (e.g., molecular beacons), sensing reagents, or donor beads. In some embodiments, the solid support is fabricated from a thermoplastic polymer (e.g., polystyrene or polypropylene). In some embodiments, a surface of the solid support is hydrophobic, hydrophilic or omniphobic. In some embodiments, the solid support is white, black, or clear or a combination thereof. In some embodiments, the solid support is opaque, transparent, translucent, or a combination thereof. In some embodiments, the substrate is conducting to allow electrochemical or electrochemiluminescent reactions and assays. In some embodiments, the substrate is an optical waveguide that allows efficient and integrated optical detection. In some embodiments, the solid support is a sensor (e.g., surface acoustic wave sensor). In some embodiments, the second topographically defined area is 2 mm or greater away from an edge of the solid support. In some embodiments, the systems and methods further comprises a detection system selected from, for example, colorimetric, fluorescent, fluorescence polarization or lifetime readings, refractive index change, and electrochemical detection systems (e.g. surface plasmon resonance detection, ring oscillator detection, etc.). In some embodiments, a single antigen is used to generated different standard curves, of differing dynamic ranges within one well.

Further embodiments of the present invention provide methods of detecting analytes (e.g., multiplexed methods) using the aforementioned systems.

Additional embodiments are described herein.

### DESCRIPTION OF THE FIGURES

FIG. 1 shows a schematic of custom microplates for the simultaneous detection of four antigens (4-plex assays) using four homogenous phase assay reagents. (a) A side view of the plate shows that each of the 96 large wells contains 4 smaller wells. (b) A top view of one of the large 96 wells illustrates one example of the spacing between the 4 smaller wells. (c) A top view of the entire custom 4-plex plate shows that the plate can be read as a 96-well or 384-well plate in commercial plate readers.

FIG. 2 shows a schematic of custom microplates for 4-plex plates whereby the plate satisfies both 96-well and 1536-well SBS standards. (a) A cross sectional side view of one large 96 well shows that each 96-well contains 4 smaller wells, which satisfy 1536-well SBS standards. (b) An aerial view of the entire custom 4-plex plate shows that the plate can be read as a 1536-well plate in commercial plate readers.

FIG. 3 shows a schematic of the slide array that is compatible with optical plate readers as well as imaging systems (a). Solution pools containing antigen are confined using a hydrophobic materials and aqueous 2-phase systems containing antibodies are subsequently applied to achieve multiplexed readouts for each confined sample (b). Physical walls replacing the hydrophobic lines.

FIG. 4 shows a comparison of singleplex and multiplex curves using custom 4 plex (384 W) plate that demonstrates minimal optical crosstalk in the custom plates.

FIG. 5 shows multiplex curves using a custom 4 plex (384 W) plate of embodiments of the present invention showing minimal optical crosstalk in custom plates.

FIG. 6 shows a comparison of IL6 singleplex curves in a thermo V-bottom 384 Plate versus a custom V-bottom 4-plex (384) plate of embodiments of the present invention.

FIG. 7 shows singleplex standard curves for 5 biomarkers generated using ATPS-AlphaLISA on thermo V-bottom 384 Well plates.

FIG. 8 shows an example of standard curves for four biomarkers generated using ATPS-ELISA on a custom slide array of embodiments of the present invention. Grey lines represent the locations of wax boundaries that prevent mixing of different standard solutions.

FIG. 9 shows lack of crosstalk in plates of embodiments of the present invention.

FIG. 10 shows sensitivity and dynamic range of plate of embodiments of the present invention.

FIG. 11 shows 4 plex assays using plates of embodiments of the present invention.

FIG. 12 shows small scale clinical trials using plates of embodiments of the present invention.

FIG. 13 shows a) multiplexing ELISA using commercially available reagents; b-c) ELISA signal area decreased as polymer concentration increased for each phase systems; d) droplet spreading.

FIG. 14 shows a) control of droplet spacing by adjusting the tip spacing; b-c) multiplexing with different droplet sizes; d) droplet spacing; e) spotting of the detection antibody on a dried well that had been treated with capture antibody and antigen; f) prevention of cross talk between detection antibodies using ATPS; g-h) assays in multiwall plates.

FIG. 15(a-g) shows a comparison of systems of embodiments of the present invention to conventional ELISA.

FIG. 16(a-c) shows detection of GVHD markers using capture antibodies.

FIG. 17 shows an example of standard curves for four biomarker generated using ATPS-ELISA.

FIG. 18 shows an array of 4-plexed dimples in a multiwall plate.

FIG. 19 shows a multiplex plate a) and sample locations in the plate b).

FIG. 20 shows the generation and use of plates with pre-dried reagents.

FIG. 21 shows a diagram of an immunoassay performed using methods of embodiments of the present invention.

FIG. 22 shows a diagram of a comparison of a singleplex immunoassay and a multiplexed immunoassay of embodiments of the present invention.

FIG. 23 shows a) partition coefficients and b-d) detection results of assays of embodiments of the present invention.

FIG. 24 shows the linear range of detection of immunoassays of embodiments of the present invention.

FIG. 25 shows the results of multiplex GVHD detection assays.

### DEFINITIONS

To facilitate an understanding of the present invention, a number of terms and phrases are defined below:

The term “assay reagents” as used herein is used in the broadest sense and may refer to freely-circulating antibody-conjugated beads, surface-tethered antibodies, nucleic acid reagents, enzymes, detection reagents, or other biomolecules.

The term “pre-equilibrated” as used herein is used in the broadest sense to refers to a two-phase system prepared such that polymer solutions are equilibrated (e.g., by composition) towards their thermodynamically most stable states prior to their intended application (e.g., immunoassay).

The term “non pre-equilibrated” as used herein is used in the broadest sense to refer to individually prepared polymer solutions that comprise an aqueous two-phase system significantly different from their thermodynamically most stable state. In some embodiments, when phases are non pre-equilibrated, the two phases of the ATPS become equilibrated within the assay system during the assay via convection and diffusion enhancing mass transport.

The term “sample” in the present specification and claims is used in its broadest sense. On the one hand it is meant to include a specimen or culture. On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

As used herein, the term “viscosity” refers to a measure of the resistance of a fluid deformed by either shear stress or tensile stress. In some embodiments, viscosity is measured in poise or centipoise (cP) units.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds. A test compound can be determined to be therapeutic by screening using the screening methods of the present invention.

### DETAILED DESCRIPTION OF THE INVENTION

The present invention relates to systems and methods for an aqueous 2-phase system droplet array and uses thereof. The present invention further relates to methods for performing multiplexed assays, chemical, and/or biological experiments and tests (e.g., immunoassays).

In some cases, individual biomarkers do not have sufficient specificity and sensitivity to act as a clinical diagnostic tool. Instead, to accurately differentiate between healthy and diseased states, simultaneous measurement of a panel of biomarkers is useful. Conventionally, multiplexed diagnostic assays are heterogeneous assays, which require multiple wash steps to separate unbound reagents from bound reagents. The most common example of a heterogeneous assay is enzyme-linked immunosorbent assay (ELISA). However, current commercially available multiplexed ELISAs have many limitations: (1) antibody crosstalk between secondary antibodies reduces assay sensitivity and (2) multiple steps make assay very time consuming, requiring as much as 4 to 10 hours to perform (Fu 2010, Ellington 2010, Kingsmore 2006, Gonzalez 2008). For example, Theilacker et al. recently demonstrated the nonspecific binding of IL-6 and IL-8 detection antibodies to TNFα capture antibodies (Theilacker 2011).

In contrast to heterogeneous assays, homogeneous assays do not require any wash steps. One example of a homogeneous assay is AlphaLISA (amplified luminescent proximity homogeneous assay). In an AlphaLISA assay, antigens are sandwiched between biotinylated detection antibodies and anti-analyte conjugated acceptor beads for 1 hour. Then streptavidin-coated donor beads are added to the reaction and incubated for 30 minutes. Upon laser excitation at 680 nm, the photosensitizing agent (phthalocyanine) within donor beads, excite ambient oxygen to a singlet state. If the donor and acceptor beads are brought within 200 nm by capture of the analyte, oxygen singlets generate a fluorescent signal in the acceptor bead, resulting in an emission signal at 615 nm. AlphaLISA assays, however, is difficult to multiplex in its current format because introduction of multiple detection antibodies as a cocktail into the solution leads to significant antibody crosstalk.

Capitalizing on the stable partitioning of antibodies and antigens reagent beads in the dextran phase of poly(ethylene glycol)-dextran aqueous two-phase system (ATPS), embodiments of the present invention provide multiplexed detection of biomarkers using AlphaLISA assay reagents. Because antibodies remain confined within the dextran phase, the multiplexed immunoassay does not exhibit antibody crosstalk. In addition, the multiplexed assays require no wash steps and results are obtained within two hours. Additionally, confinement of antibodies and reagent beads in 2 μL assay droplets reduces assay cost by 25-fold.

### I. Solid Supports

In some embodiments, the present invention provides solid supports (e.g., assay plates or other support) for performing multiplexed aqueous two-phase immunoassays. In some embodiments, supports are, for example, microplates, slides, dishes, cartridges, CD assay platforms, electrochemiluminescent substrates, electrodes, or other rigid or flexible substrate, used to form an array of discrete sample droplets on surfaces and in wells.

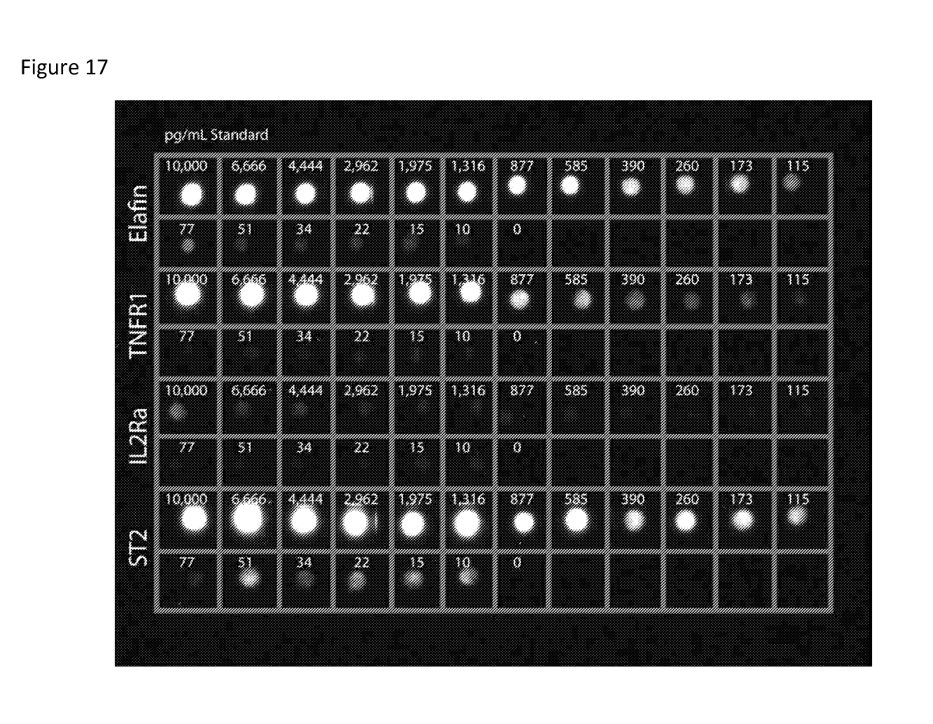

In some embodiments, plates are customized for the multiplex assay format. For example, in some embodiments, dividers are added to plates using any suitable method such as wax pencil (See e.g., FIG. 17). In some embodiments, dividers are physical or topgraphical barriers (e.g., walls, dimples, etc.). In other embodiments, dividers are chemical (e.g., hydrophobic) barriers such as wax.

The dividers prevent samples from merging and allow multiple detection spots to be placed within each sample pool (e.g., well of a multiwall plate or plastic dish). Individual sample areas are not limited to a particular size. Any size that provides adequate space for performing an assay may be utilized. In some embodiments, sample areas are from 0.02 to 0.5 cm² (e.g., 0.04 to 0.25 cm²). In some embodiments, dividers completely divide separate solutions whereas some dividers are smaller dividers that are meant to keep the heavier ATPS phase separated but to keep the specimen-containing lighter phase all connected.

In some embodiments, hydrophobic tape stencils generated by, for example, packing tape, are utilized to create sample areas in custom array plates.

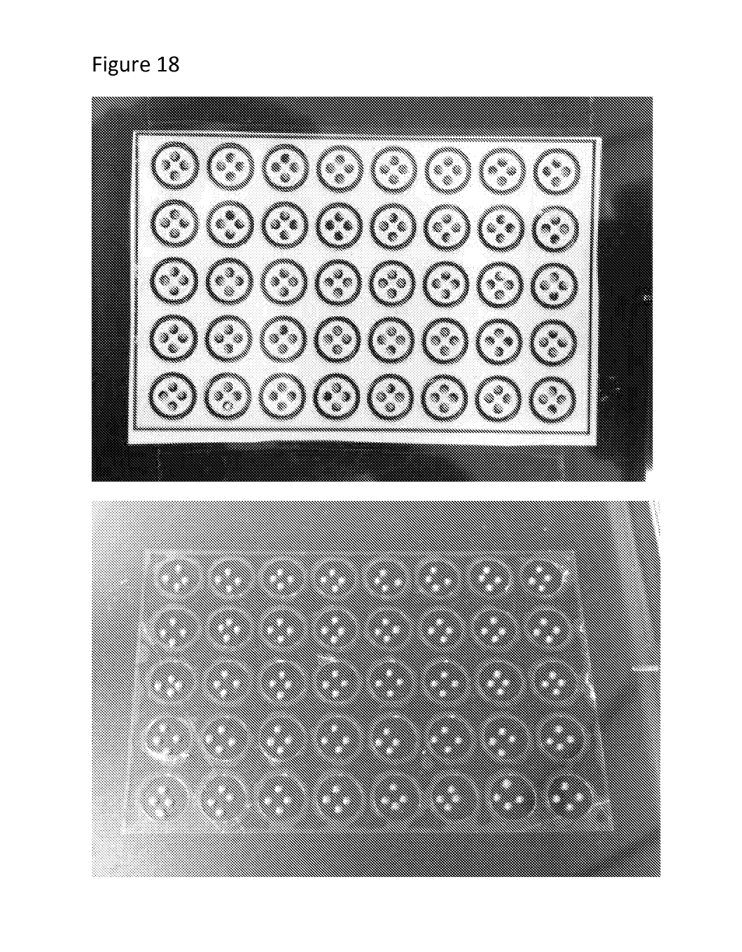

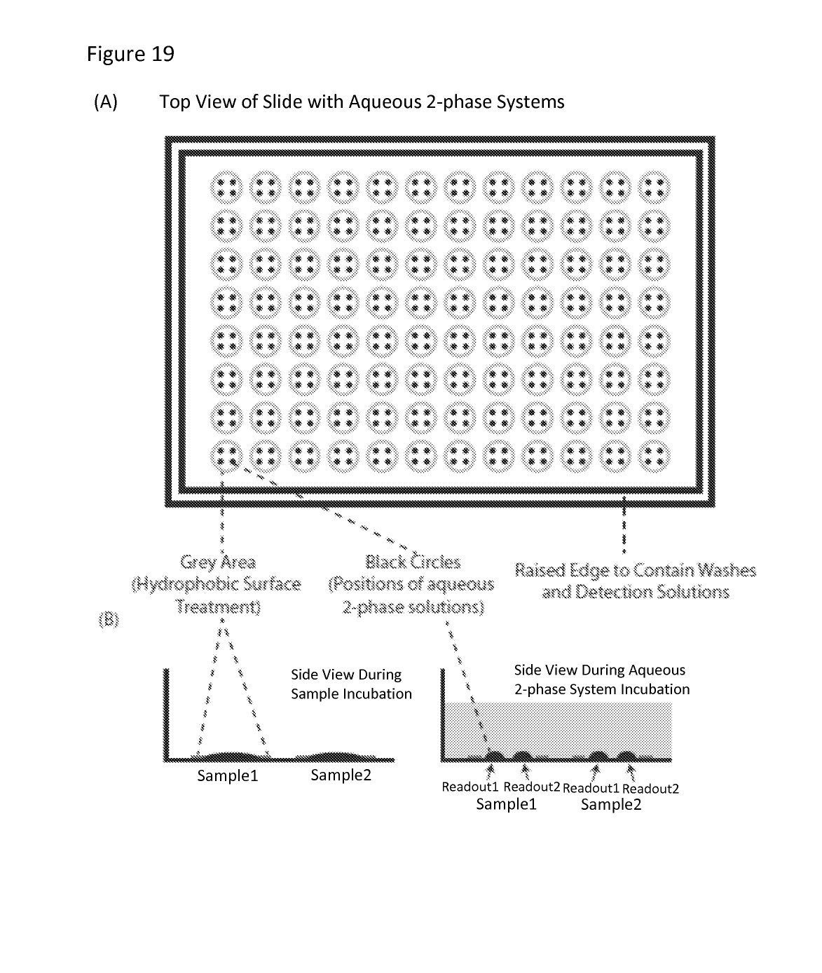

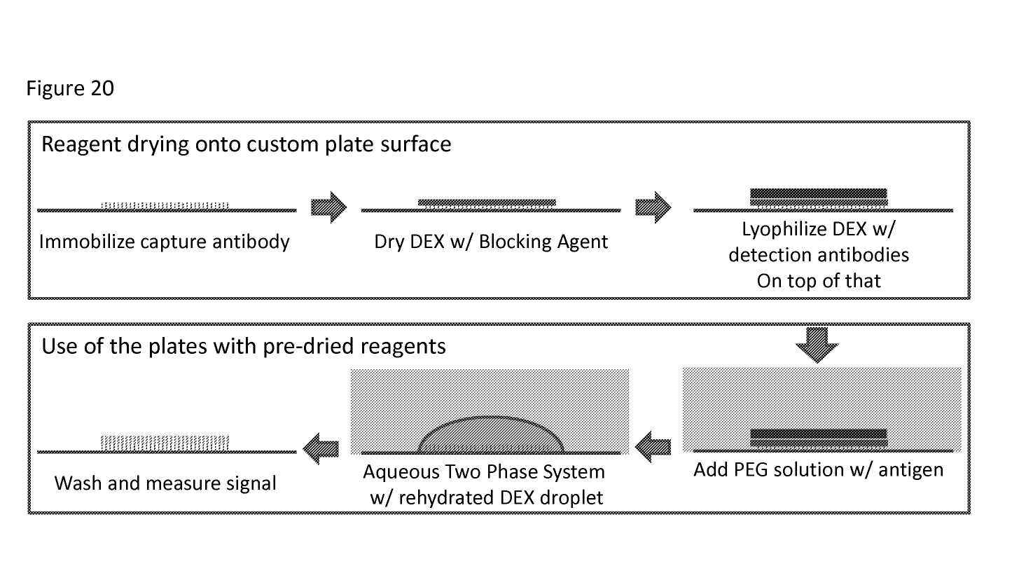

In some embodiments, plates contain dimples (e.g., to align capture and detection patterns). In some embodiments, dimples are generated by drilling or injection molding. In some embodiments, other topological features are incorporated, for example, pillars, notches, etc. to increase surface are or enhance optical guidance. FIG. 18 shows an example of a custom array plate comprising dimples.

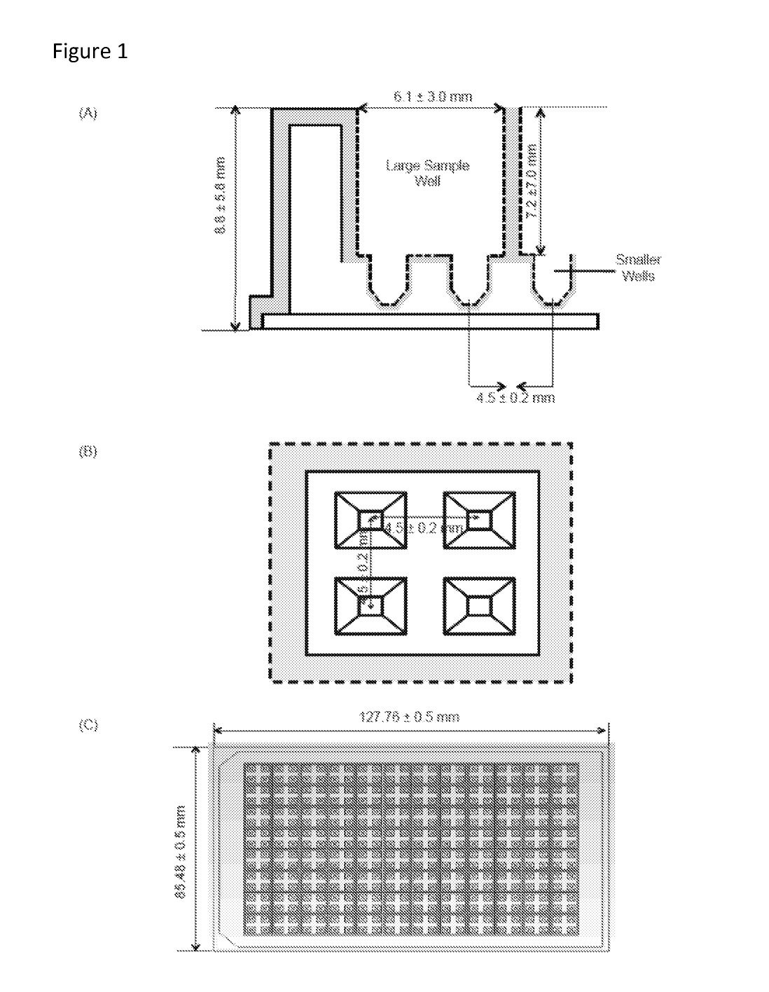

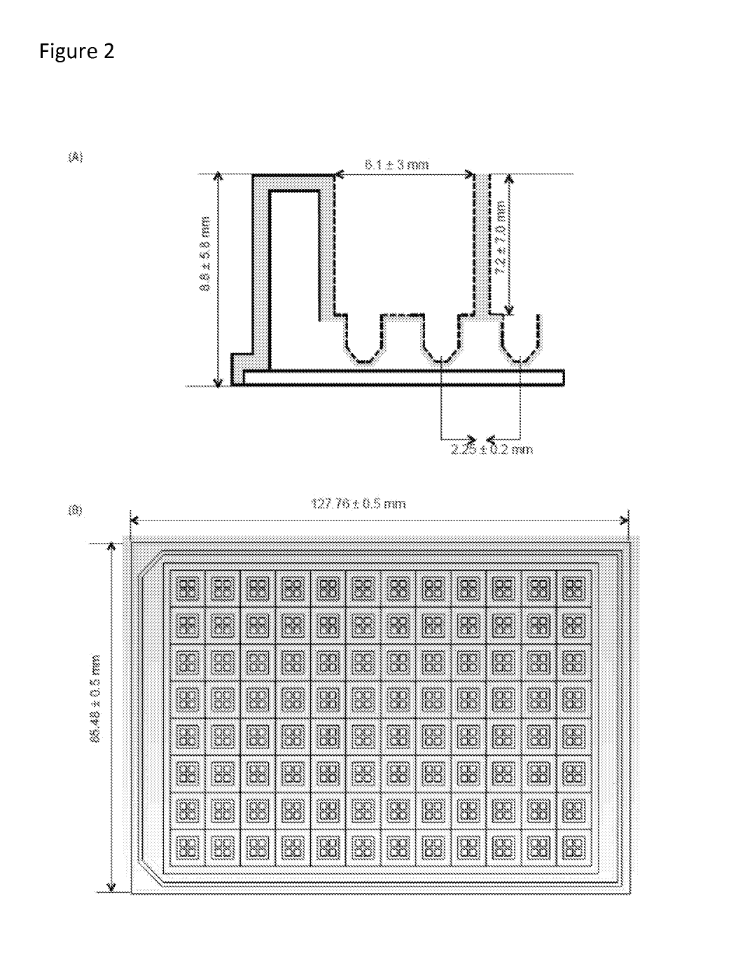

In some embodiments, custom plates for multiplex bead-based assays are designed. In some embodiments, plates are fabricated with a rigid, white, opaque material in a printer. FIGS. 1-2 show an example of such plates. In some embodiments, to identify the optimum design of the multiplex plate, different variations of the microplate dimensions and plate material are tested. One major design consideration is to eliminate optical crosstalk, thereby avoiding the need to create a complex algorithm to accurately quantify the chemiluminescent readout per well. Wells with a strong chemiluminescent signal can mask weaker signals in neighboring wells. To ensure the detection limits obtained in singleplex ATPS assays is comparable to the detection limits obtained in multiplex format, optical crosstalk obtained in multiplex plates made from various plate materials is compared. In some embodiments, multiplex plates are all white, however, use of all black or black-walled plates can decrease well-to-well optical crosstalk. In some embodiments, supports are opaque, translucent, transparent or a combination thereof. To investigate this, the all-white plate is compared to all-black plates, black DEX wells with white PEG walls, and white DEX wells with black PEG walls. The optical crosstalk obtained in each plate configuration is calculated in order to identify the optimum plate material.

To evaluate optical crosstalk in each multiplex plate, one sample with very bright chemiluminescent signal (approximately 100 times the background signal) and another sample with very low chemiluminescent signal are used. The low activity sample will have about 5 times the background signal. Individual well crosstalk is defined by the following:

Individual well crosstalk=(Average counts of 8 empty DEX wells around the well with high-activity or low-activity sample−Average counts of surrounding 16 empty DEX wells)/(Average of the count of the active well, containing the low or high-activity sample).

A second parameter, cumulative crosstalk, is quantified to account for when a high-activity and low-activity sample are in neighboring DEX wells.

Another design consideration is the reduction of sample volume required to fill the PEG well. By designing custom multiplex plates where the DEX wells are arranged in the standard 1536 well spacing (2.25 mm center-to-center well spacing), the size of the PEG wells can be reduced to as much as 5.5 mm×5.5 mm. To minimize optical crosstalk, the well plate can be read using a 3456 aperture. Reducing the area of the PEG wells reduce the volume of sample required to perform the multiplex assays.

Plates or solid supports may be constructed using any suitable material (e.g., including but not limited to, glass, paper, hydrogels, tapes, cloth, yarn, thread, or a thermoplastic polymer such as polystyrene, polypropylene, or conducting surfaces such as indium tin oxide (ITO), gold, platinum, or conducting polymer). In some embodiments, supports comprise a coating that renders the surface hydrophobic, hydrophilic, omniphobic, or a combination thereof. In some embodiments, the solid support is a sensor such as a surface acoustic wave sensor.

### II. ATPS

As described herein, embodiments of the present invention utilize aqueous two-phase systems (ATPS). Aqueous two phase systems are described, for example, in WO 2012/040473 and WO 2011/116256, each of which is herein incorporated by reference in its entirety. Briefly,

In some embodiments, the present invention provides multi (e.g., two) phase solution based microarrays and uses thereof. The present invention is not limited to particular components of the microarray. In some embodiments, the components are aqueous polymers or other solutes (e.g., salts such as sodium carbonate, PEG or and phosphates). Preferred polymers are those that form an aqueous two phase system (ATPS) at a wide range of temperatures. Examples of suitable polymers include, but are not limited to, polyethylene glycol (PEG), polyvinyl alcohol (PVA), hydroxypropyl dextran (HPD) or dextran (DEX), and combinations of other polymers such as DEX-methylcellulose, DEX-polyvinyl alcohol, PEG-DEX sulfate, polyvinyl alcohol-DEX sulfate, hydroxypropyldextran-DEX, and DEX sulfate-methylcellulose. Other specific, non-limiting examples include but are not limited to, DEX T10, PEG 35,000, DEX T40, PVA 105,000, HPD 500 and DEX T500. In some embodiments, DEX polymers are used in concentrations above 1% to suppress background noise and enhance sensitivity.

In some embodiments, charged polymers like diethylaminoethyl (DEAE) dextran or carboxymethyldextran are mixed with the DEX phase to promote charged analytes (e.g., cytokines) partitioning or localizing to the DEX phase.

In other embodiments, ATPS that exhibit variable phase separation with temperature are utilized. In some embodiments, such systems utilize low or high molecular weight polymers.

In some embodiments, a first solution comprising a first polymer and detection reagents for detection of an analyte of interest or a test sample (e.g., comprising the analyte of interest) is dispensed onto a solid support. In some embodiments, a second solution comprising a second polymer detection reagents for detection of an analyte of interest or a test sample (e.g., comprising the analyte of interest) is dispensed onto the first solution, thus forming an aqueous two phase system. In some embodiments, a transport component (e.g., an array of slot pins) is then used to transfer the second solution onto the array comprising the first solution. For example, in some embodiments, a multiplex dispenser that allows different materials to be added to different spots on the array is utilized. In some embodiments, the dispenser is a plurality of pins or other dispensing components affixed to a single transport component. In some embodiments, the transport component is automated.

In some embodiments, aqueous two phase polymer formulations that give relatively less viscous phases (e.g., less than 10 cP) that enable reagents to diffuse more freely and enhance detection sensitivity are used. Examples, include, but are not limited to DEX T10 that is less viscous than an ATPS with higher molecular weight DEX but also is stable and not precipitating like DEX T3.5.

In some embodiments, aqueous two phase systems with relatively high viscosity (e.g., more than 10 cP) that provide higher stability in terms of response to convection due to higher viscosity are utilized. Examples include, but are not limited to, an ATPS with DEX T40 that gives a higher viscosity ATPS.

In some embodiments, detection reagents (e.g., antibodies) are bound to a particle or are in a suspension. Examples include, but are not limited to, dextran-coated microspheres, a proximity bead assay reagent or a molecular beacon reagent.

In some embodiments, the two-phase system is prepared such that polymer solutions are equilibrated (e.g., by composition) towards their thermodynamically most stable states prior to their intended application (e.g., assay). In other embodiments, individually prepared polymer solutions comprise an aqueous two-phase system significantly different from their thermodynamically most stable state (e.g., non-equilibrated). In some embodiments, when phases are non pre-equilibrated, the two phases of the ATPS become equilibrated within the assay system during the assay via convection and diffusion enhancing mass transport.

In some embodiments, the capture antibody, detection antibody, polymers used for aqueous two phase formation, blocking agents, enzymes, and other reagents are be pre-spotted on the custom plates in a dried or lyophilized form. In these reagent pre-spotted plates/slides, addition of an appropriate aqueous solution leads to rehydration and reformation of the aqueous two phase system droplets.

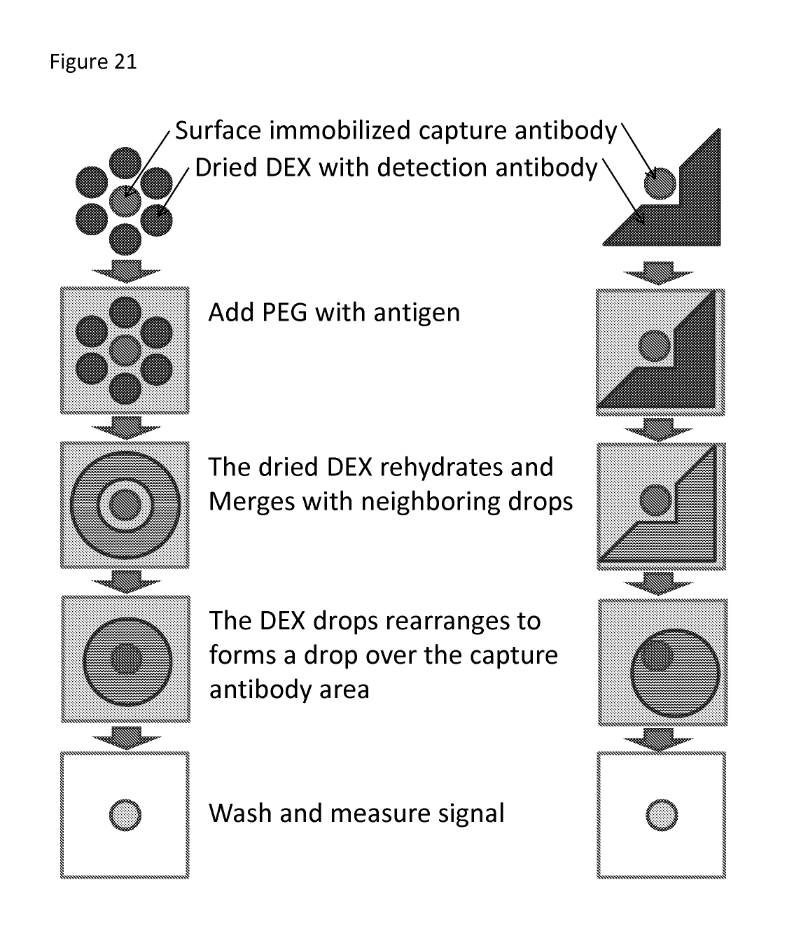

Examples of a plate where the primary antibody is pre-coated and immobilized onto the surface of the custom plate, and a DEX-rich phase solution with the capture antibody is lyophilized on top of the capture antibody immobilized surface (schematic shows side view) are shown in FIG. 21. There may also be multiple layers of dried reagents such as a capture antibody surface or a blocking protein containing dried DEX layer followed by lyophilized DEX phase with capture antibodies positioned on top. The PEG phase may also be pre-dried onto the plates so that users only need to add regular aqueous solutions without PEG or DEX or other aqueous two phase forming components. The pre-dried reagents can be polymers other than PEG or DEX that can form ATPS.

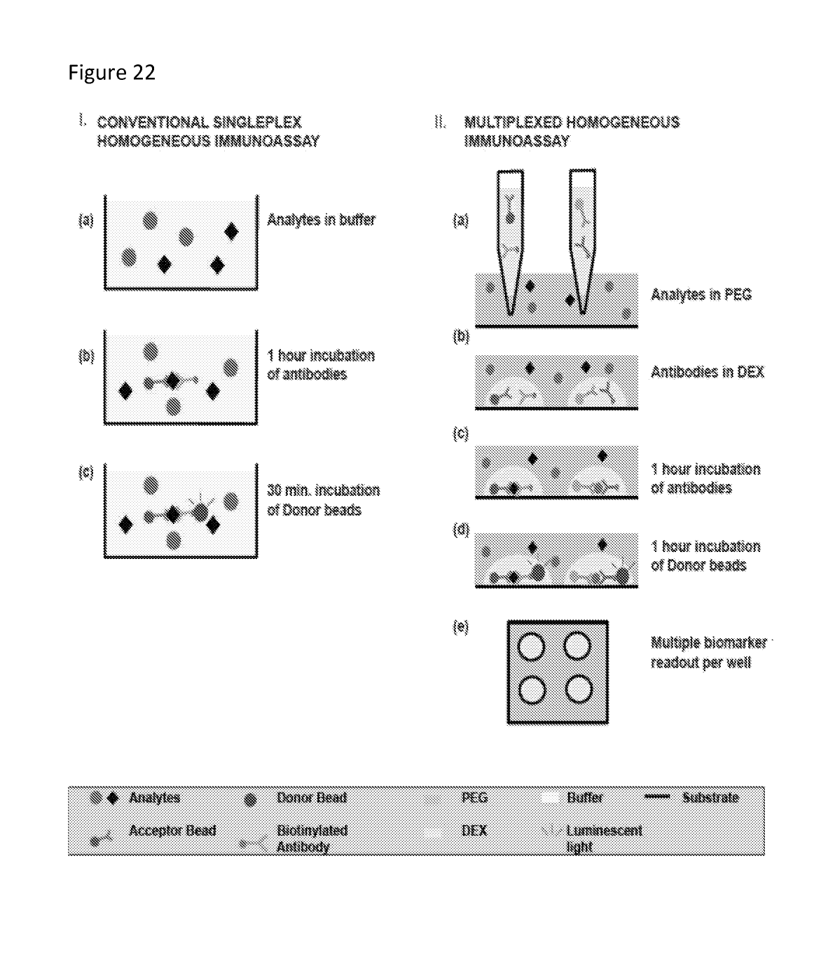

In some embodiments, the pattern of dried reagents formed is varied. For example, a capture antibody coated surface may be surrounded by a ring of dried DEX droplets that include the detection antibody (See e.g., FIG. 22). Upon rehydration, the outer ring of DEX droplets can merge and then collapse into a single larger droplet that exposes the capture antibody site now with a solution of DEX—detection antibody. One may use other shapes of the dried heavier phase (e.g. DEX phase) such as acute angles that also change shape upon rehydration with the lighter phase of the aqueous two phase system (e.g. PEG).

### III. Assays

The multiplex solution based assays and plates of embodiments of the present invention find use in a variety of applications. In some embodiments, the arrays are utilized in performing assays (e.g., diagnostic, drug screening, or research assays).

In some embodiments, the first solution comprises a first polymer and a reagent for detection of an analyte and the second solution comprises a second polymer and the test sample suspected of containing the analyte. In other embodiments, the first solution comprises a first polymer and a test sample suspected of containing the analyte and the second solution comprises a second polymer and reagents for detecting the analyte. In some embodiments, the first solution is applied to a solid surface. The second solution is then selectively applied on top of the first solution, forming 2-phase solution arrays. Interaction of the reagent specific for the analyte and the analyte is then detected using any suitable method.

The present invention is not limited to a particular analyte. Exemplary analytes include, but are not limited to, antigens, antibodies, nucleic acids, proteins (e.g., biologically relevant proteins), small molecules, hormones, receptors, ligands and the like. For example, in some embodiments, assays detect cytokines or biomarkers such as HGF, ST2, TNFR1, TNFα, IL-8, IL-6, IL-2Rα, MIG, CRP, and elafin.

In some embodiments, the analyte sample is an antigen-containing buffer solution, animal or human blood plasma or serum specimen, or animal or human saliva.

The present invention is not limited to a particular detection reagent. Any detection reagent that specifically interacts (e.g., binds) to an analyte of interest and partitions selectively to one of the two aqueous phases is suitable. Exemplary detection reagents include, but are not limited to, antibodies, receptors, ligands and the like.

Embodiments of the present invention are illustrated with immunoassays. However, the present invention is not limited to immunoassays. One skilled in the art recognizes that the systems and methods described herein are applicable to any number of biological or other assays.

Exemplary immunoassays are described in Examples 1 and 2 below (e.g., lyophilized capture and/or detection antibody and bead based assays).

Interaction of an analyte and a detection reagent specific for the analyte are detected using any suitable method. In some embodiments, additional reagents are utilized in diagnostic assays (e.g., reporter molecules, chemical detection reagents, fluorescent reagents and the like).

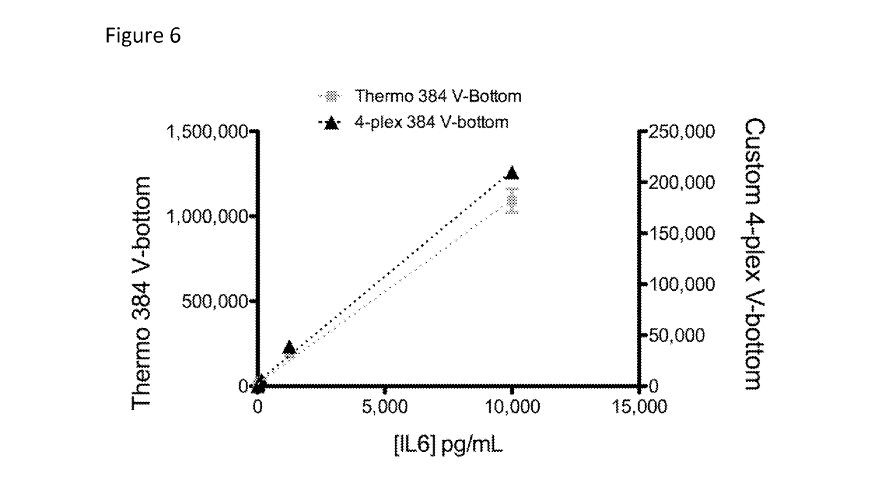

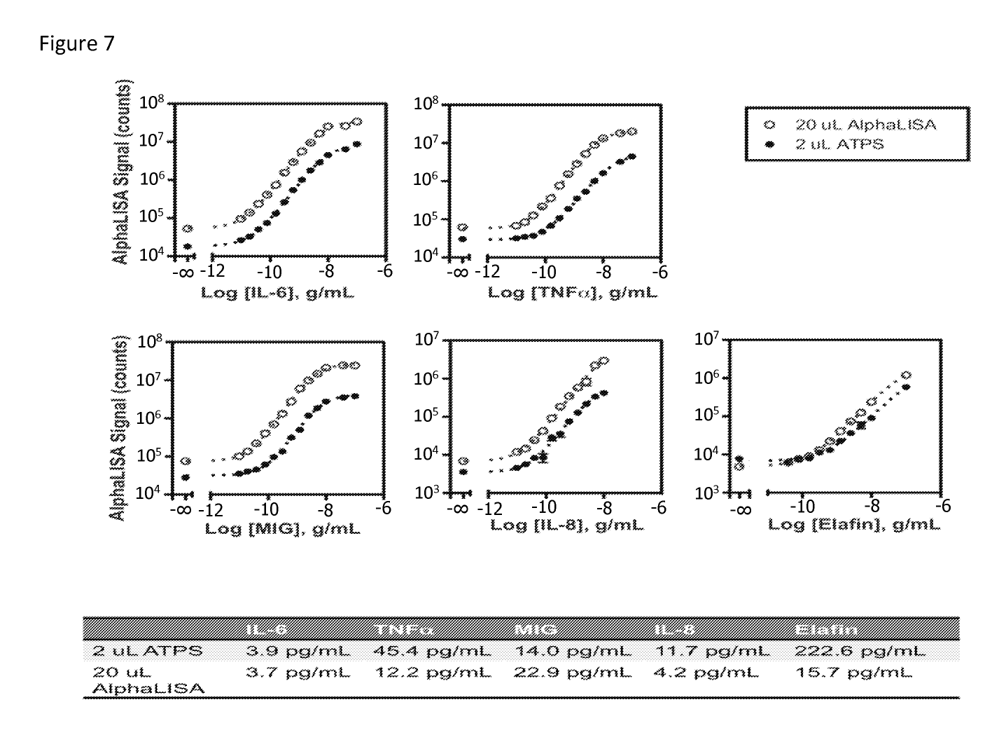

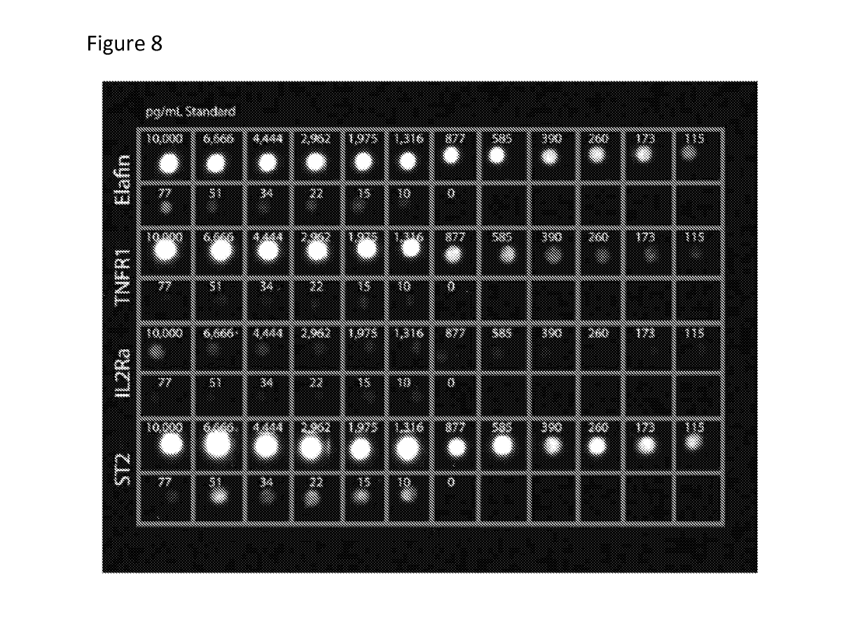

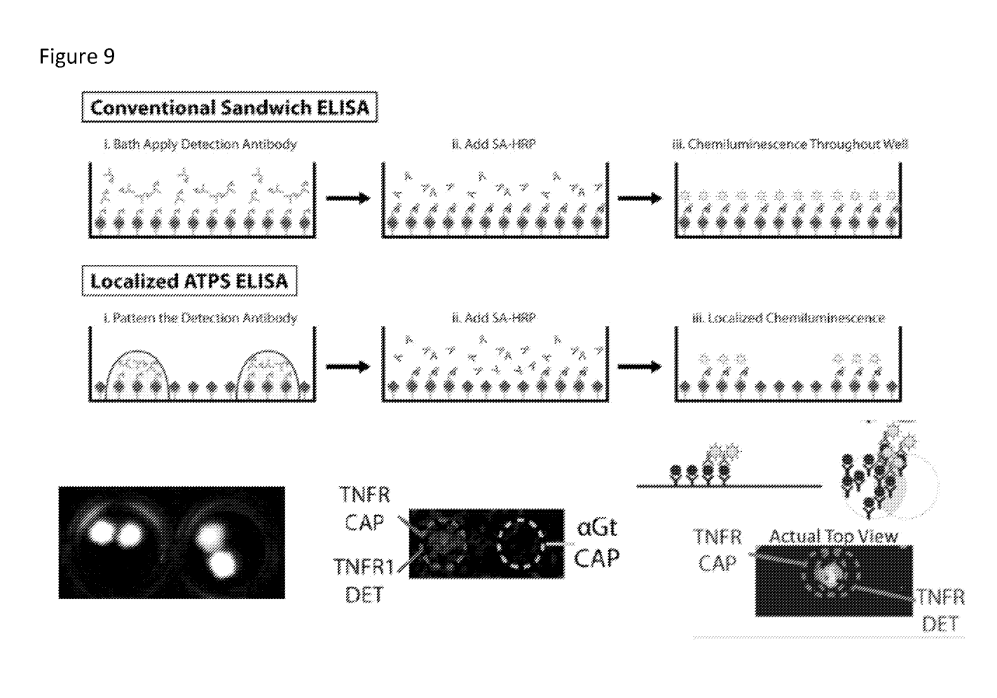

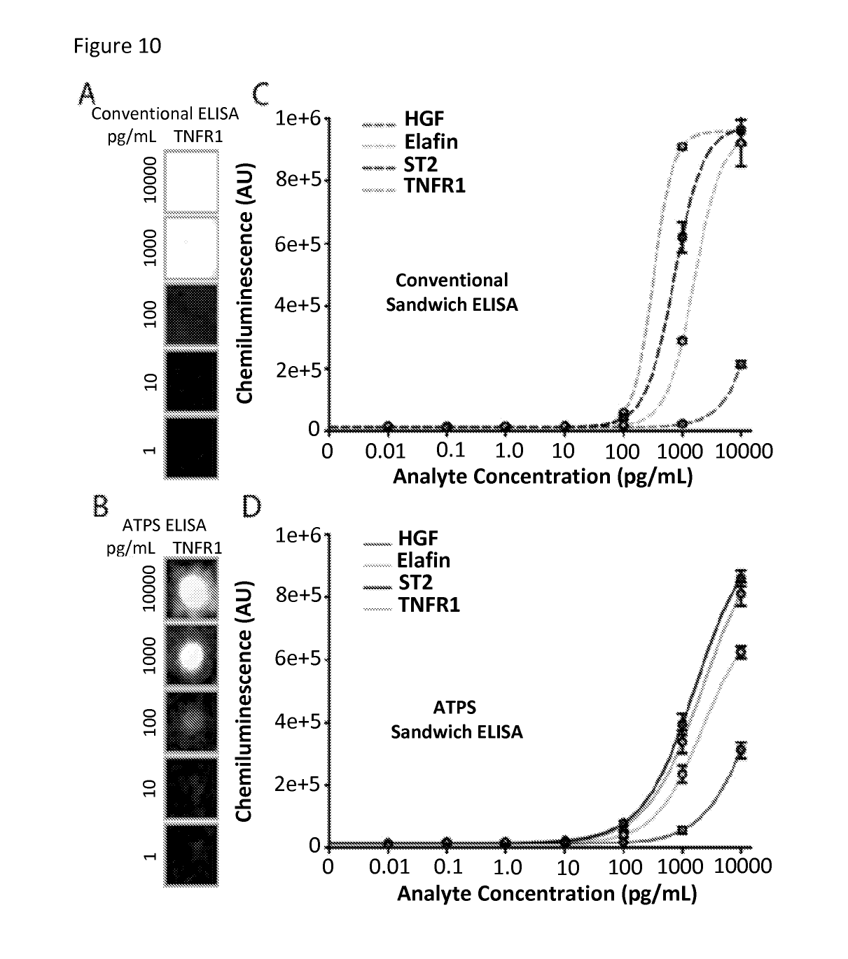

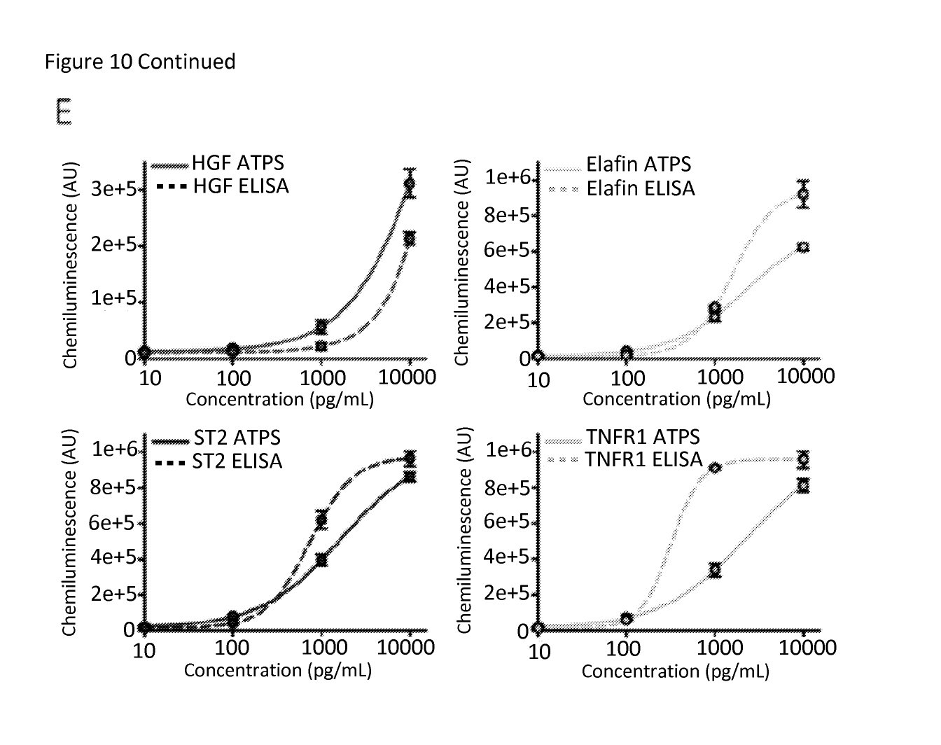

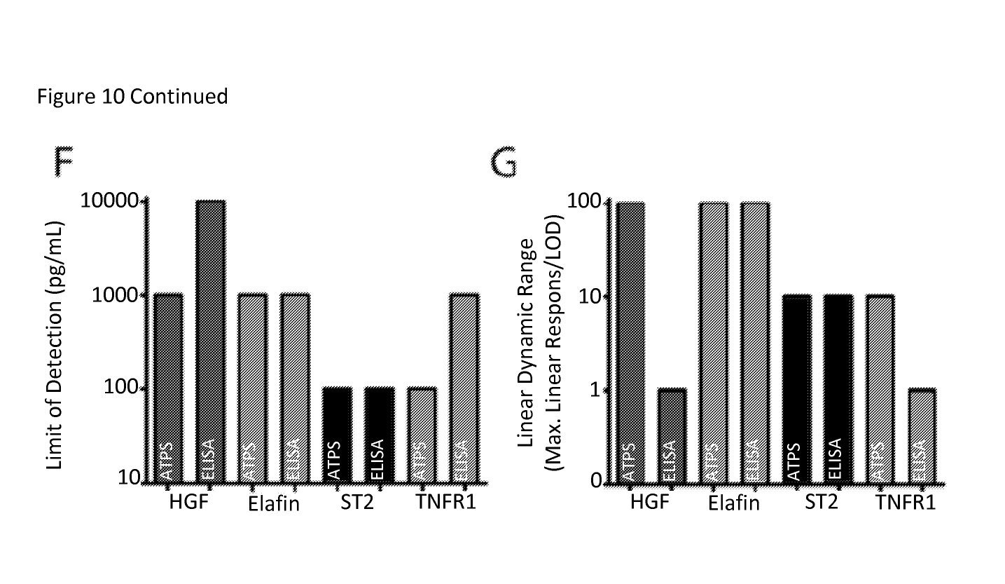

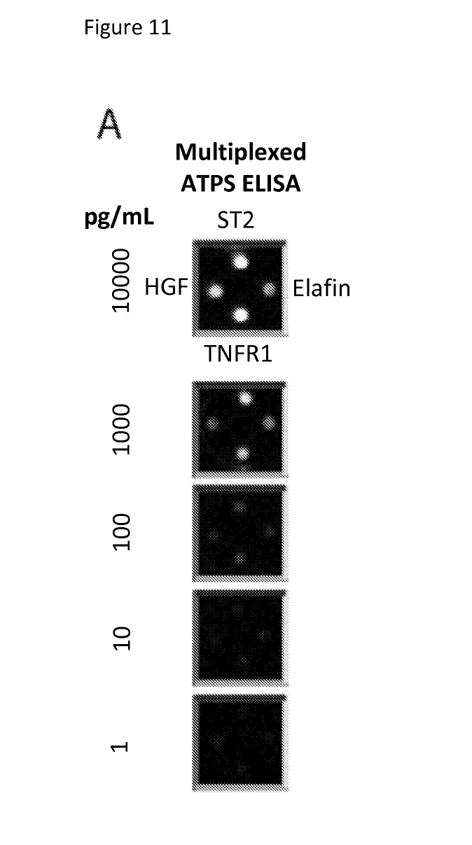

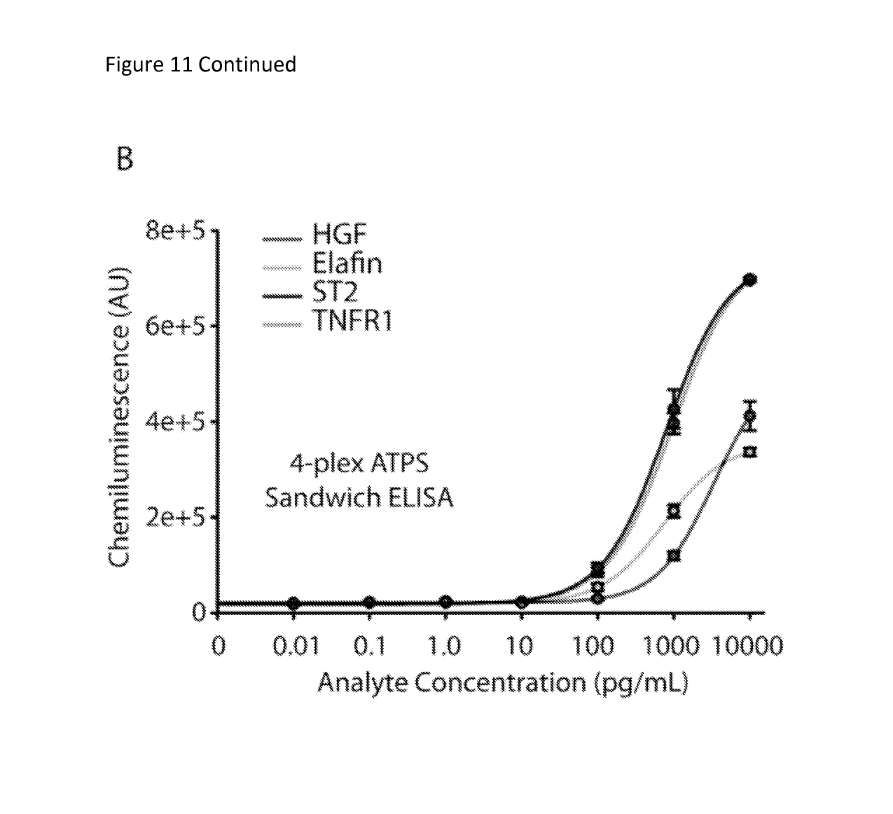

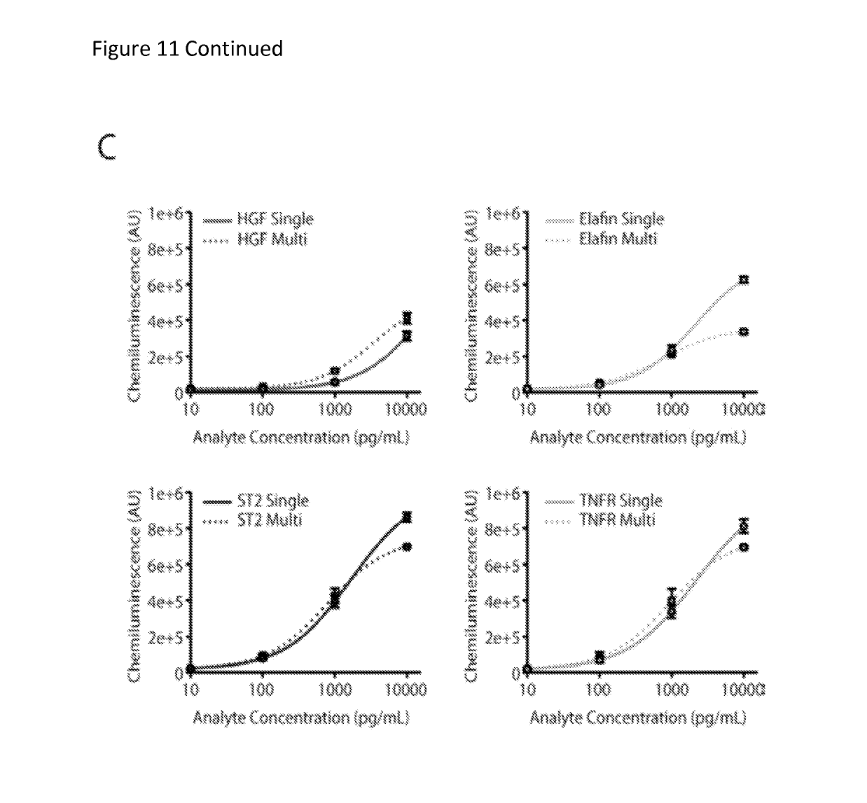

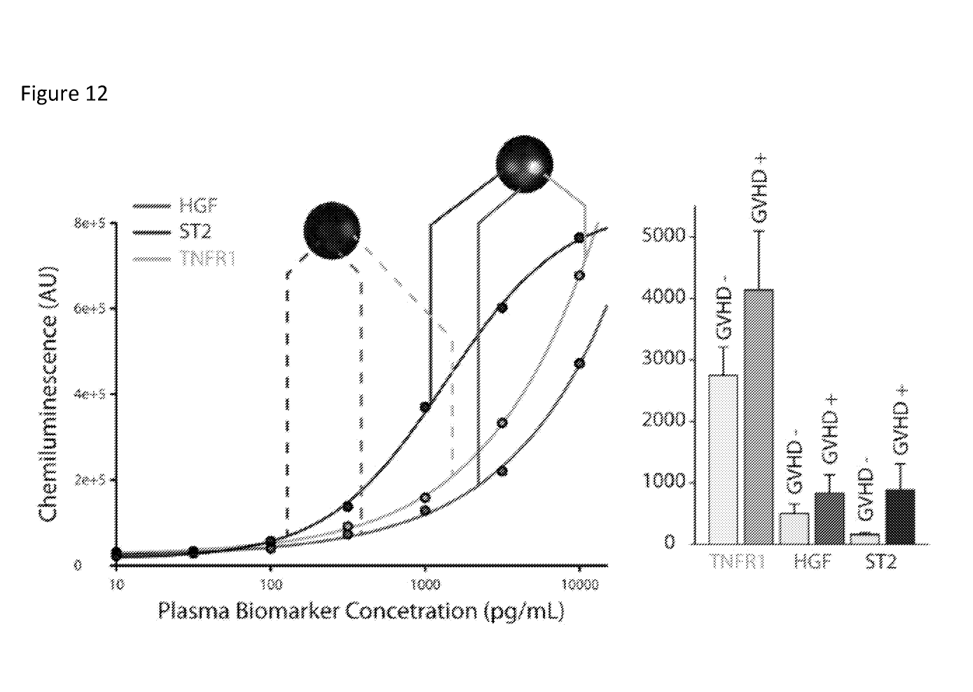

In some embodiment, a detection apparatus (e.g., a fluorometer, spectrometer, camera, etc.) is utilized in the detection of an interaction between the analyte and the detection reagent. In some embodiments, the detection apparatus described herein (e.g., in FIG. 6) is utilized. In some embodiments, detection methods include, but are not limited to laser induced luminescence, FRET (fluorescence resonance energy transfer), fluorescence polarization, transmittance, fluorescence anisotropy, raman spectroscopy or color change. In some embodiments, the optical setup includes a laser diode for reagent excitation and a CCD or PMT camera for emission signal detection.

In some embodiments, assays and read out is performed in a high throughput manner. In some embodiments, high throughput methods are automated. In some embodiments, multiplex assays are performed for the simultaneous detection of multiple analytes.

The present invention further provides systems and kits comprising the solution arrays described herein. In some embodiments, systems and kits comprise multiple solutions for forming arrays, reagents (e.g., antibodies and/or antigens), transport components (e.g., robotics), and components for read out of signal from assay results, including analysis software. In some embodiments, kits further comprise additional component useful, necessary, or sufficient for performing and analyzing the results of the methods described herein (e.g., including, but not limited to, buffers, control antigens, control antibodies, etc.).

In some multiplex applications, a 384 well format is utilized, although smaller or larger well combinations such as 96 well plates each with 16 wells in a 1536 well spacing format or 384 well plates each with 4-plex wells in 1536 well spacing format etc are suitable. The heights of the walls, the slopes and shapes can take on a range of values as long as the antigen phase reaches over all smaller antibody reagent droplets and the smaller antigen reagent droplet can be kept separate without mixing. In some embodiments, the plates comprise physical walls. In addition, the smaller droplets within each well can be confined using walls or depressions.

The array spacing is not limited to a particular format. Examples include but are not limited to the 96, 384, 1536 formats. The custom wells and slides and flexible tape roll based cartridges of embodiments of the present invention share the advantage of the ability to have a physical wall or hydrophobic material barrier or other way to confine a larger analyte sample solution and within this larger sample solution have smaller physical barriers that allow smaller aqueous two phase system analysis droplets to be arrayed therein. In some variations of the design, aqueous two phase droplets are directly spotted or printed into the larger analyte sample solution with or without predrying or lyophilizing.

### Experimental

The following examples are provided in order to demonstrate and further illustrate certain preferred embodiments and aspects of the present invention and are not to be construed as limiting the scope thereof.

### EXAMPLE 1

### Methods

### Reagents

ATPSs were created from DEX solutions of various molecular weights and concentrations (10,000 kDa and 500,000 kDa (Pharmacosmos), and 40,000 kDa (Sigma)), with 35,000 kDa PEG of various concentrations (Sigma). Phycoerythrin-conjugated mouse anti-human CD184 IgG2a (cat #555974, BD Pharmingen) and 500,000 kDa fluorescein-dextran (cat #D7136, Molecular Probes) were used to assess droplet stability and antibody localization over time. ELISA kits for human sTNF R1 (cat #DY225), human IL-2 Rα (cat #DY225), human ST2 (cat #DY523), human trappin-2 (cat #DY1747), and human HGF (cat #DY294) were purchased from R&D Systems and SuperSignal ELISA Femto Substrate (cat #37074) was purchased from Thermo Scientific.

### ELISA Protocol

Manufacturer specifications for ELISA assays were followed, with several modifications. Briefly, polystyrene plates of various formats were coated with either a mixture of capture antibodies or separate capture antibody spots reconstituted in PBS. The plates were then wrapped in Parafilm to limit evaporation and stored at 4° C. overnight. Plates were washed five times with wash buffer (PBS containing 0.05% Tween-20), and blocked with 3.0% casein for 1 h. After blocking, the plates were washed five times and incubated with sample solutions for 2 h. Following antigen application, plates were washed five times and incubated for 2 h with detection antibody either in a traditional ELISA or ATPS-ELISA format. For traditional ELISA, detection antibodies were applied according to manufacturer specifications. For ATPS-ELISA, the plate was first filled with a solution of PBS containing PEG and 0.1% casein. DEX droplets containing the appropriate biotinylated detection antibody were then pipetted into the PEG. Following detection antibody incubation, the plates were washed eight times followed by incubation with streptavidin-conjugated horseradish peroxidase for 1 h. Plates were washed five times and incubated with SuperSignal ELISA Femto Maximum Sensitivity Substrate (37074, Thermo Scientific). Chemiluminescent signal was detected using either a ChemiDoc XRS (BioRad) or FluorChem M (Protein Simple) imaging system.

### Plate Fabrication

The effect of surface charge on antibody binding was tested for glass and polystyrene surfaces that were either plasma oxidized for 2 minutes at 50 W using a Covance-1MP plasma oxidizer (Femto Science) or left untreated. To reduce the volume of sample required to perform the assay several methods were used to customize the plates. Sample volumes were contained either by inscribing wax markings on the plate surface with a wax Sharpie pencil, or by adhering a hydrophobic tape stencil to the surface. To aid in localization of capture antibody spots and DEX droplets containing detection antibody a dimple array was fabricated as a feature of the plate surface.

### Antibody Application

Capture antibodies were applied to specific regions of the plates in 2 μL of PBS. To prevent dehydration wet Kim Wipes were placed in each dish and the dishes were sealed with Parafilm. After an incubation period the entire plate was washed, blocked, and the wax or tape regions separating the sample areas were sufficiently dried to prevent mixing due to residual wash buffer. Care was taken not to dehydrate the antibody treated regions. After sample incubation PEG solution containing the antigen or sample was applied and dextran droplets containing the detection antibodies were dispensed on top of the original capture antibody patterns.

### Plate Fabrication

Custom plates were fabricated using several methods. The first method involved drawing wax dividers on the surface of the polystyrene plate using a wax Sharpie marking pencil. The hydrophobic wax markings prevented separate plasma samples from merging, but allowed multiple detection antibody spots to be placed within each sample pool. Dimensions ranged from 0.25 cm2× to 0.04 cm2 for individual boxes with wax edges that contained the plasma samples. Arrays of up to 15×15 boxes could be incorporated onto each plate.

The second method utilized hydrophobic tape stencils created by xurographic cutting of 3M packaging tape. The advantage of this method was that it allowed well spacing and geometry to be precisely controlled, allowing the plate fabrication procedure to be more reproducible between experiments. Briefly, circular (3-7 mm diameter) patterns were cut into the tape. The circles were then removed and the remaining tape stencil secured to a polystyrene Petri dish. Samples were retained on the dish within the cutout circular regions of the tape. An additional consideration in the design of our plates was the incorporation of dimples on the plate surface. The purpose of the dimples was to provide sites to align capture and detection antibody patterns and to improve the stability of dextran droplets containing the detection antibodies. The dimples (˜1 mm in diameter, ˜0.5 mm or less deep, and ˜2 mm edge-to-edge) were initially fabricated by drilling into the polystyrene surface using a drill press. Future generations of these plates will be fabricated using injection molding. Additional modifications possible to apply to the capture antibody region include, for example, using topographical features (pillars, notches, and other roughness features) to increases surface area or enhance optical guidance.

### Plasma Samples

Heparinized plasma samples were collected from patients who received allogeneic bone marrow transplantation at the University of Michigan between 2000 and 2010. Plasma samples were collected under protocols approved by the University of Michigan Institutional Review Board and stored at the University of Michigan. To mitigate effects associated with mismatch between the standard diluent and plasma sample several approaches were taken. First, an appropriate dilution of plasma was determined for each biomarker. Next, spike and recovery and linearity of dilution for the standard and sample diluents containing FBS, BSA and/or healthy pooled plasma were investigated. Finally, a standard diluent of healthy pooled plasma containing 1% FBS and a sample diluent of 1% FBS were selected and used for all patient sample analyses.

### Results

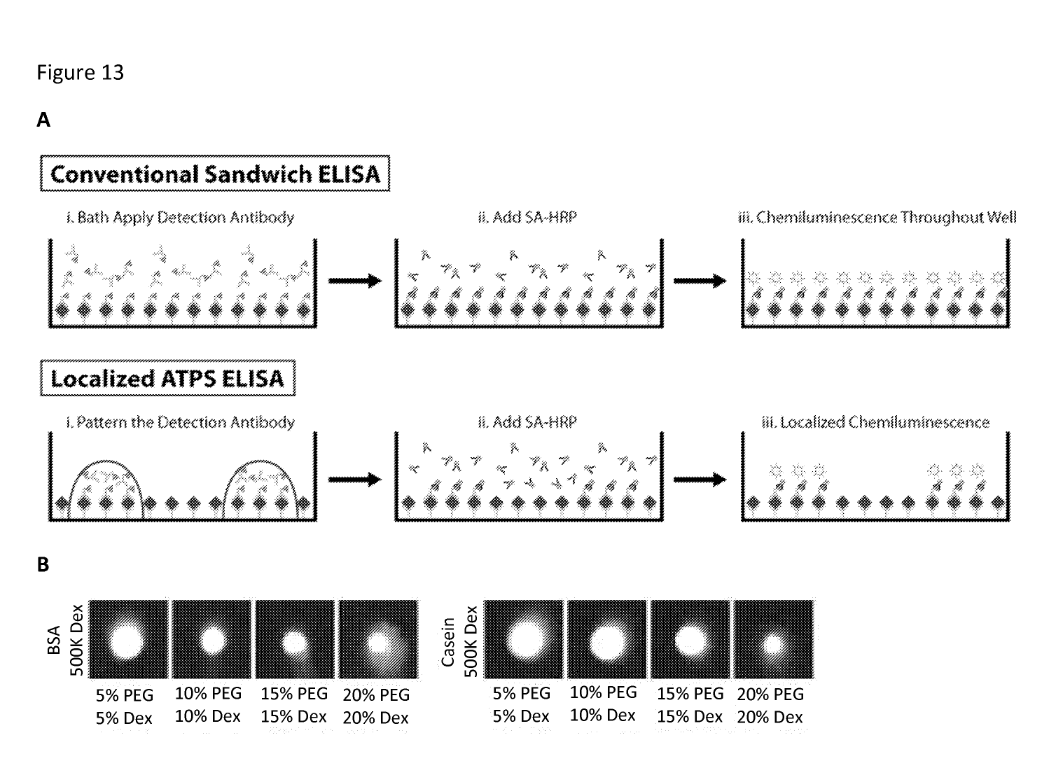

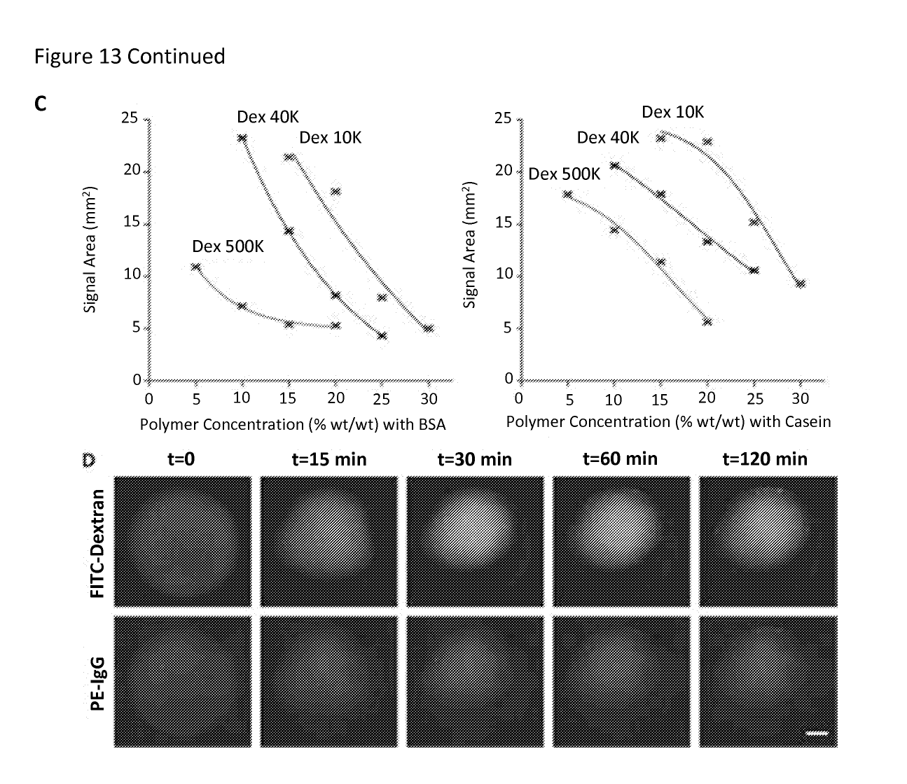

Conventional sandwich ELISA is carried out with three main steps: immobilizing the capture antibody, capturing the antigen, and antibody-mediated detection of the antigen. ATPS-ELISA does not depart from these steps, but rather provides a simple method for multiplexing ELISA using commercially available reagents (FIG. 13A). The only modification to the conventional assay workflow is placement of the ATPS on the assay plate.

### Optimization of ATPS-ELISA Spotting

ATPSs can be formed using a variety of polymers that separate from each other when mixed above threshold concentrations. The physicochemical properties of the phase system formed by these two polymers depend on polymer molecular weights and concentrations. ELISA signal area decreased as polymer concentration increased for each phase systems tested (FIG. 13B-C). This was due to increased interfacial tension and viscosity at higher polymer concentrations that prevented DEX droplets from spreading. This was supported by observation of PE-IgG (phycoerythrin-Immunoglobulin G) retention in the dextran droplets. PE-IgG was completely retained within the dextran droplet over the course of a 2 h incubation; therefore, the larger spot size at lower polymer concentration is not due to diffusion of antibodies out of the DEX droplet but instead due to increased droplet spreading (FIG. 13D). The presence of different blocking agents had only subtle effects on ELISA spot geometry and intensity. Casein was used as a blocking agent because it is more cost effective than bovine serum albumin (BSA) and produces slightly more uniform ELISA signal in DEX treated areas. The 20% DEX 500K/20% PEG 35K system produced the smallest signal area for a given DEX volume, so this was selected for further characterization.

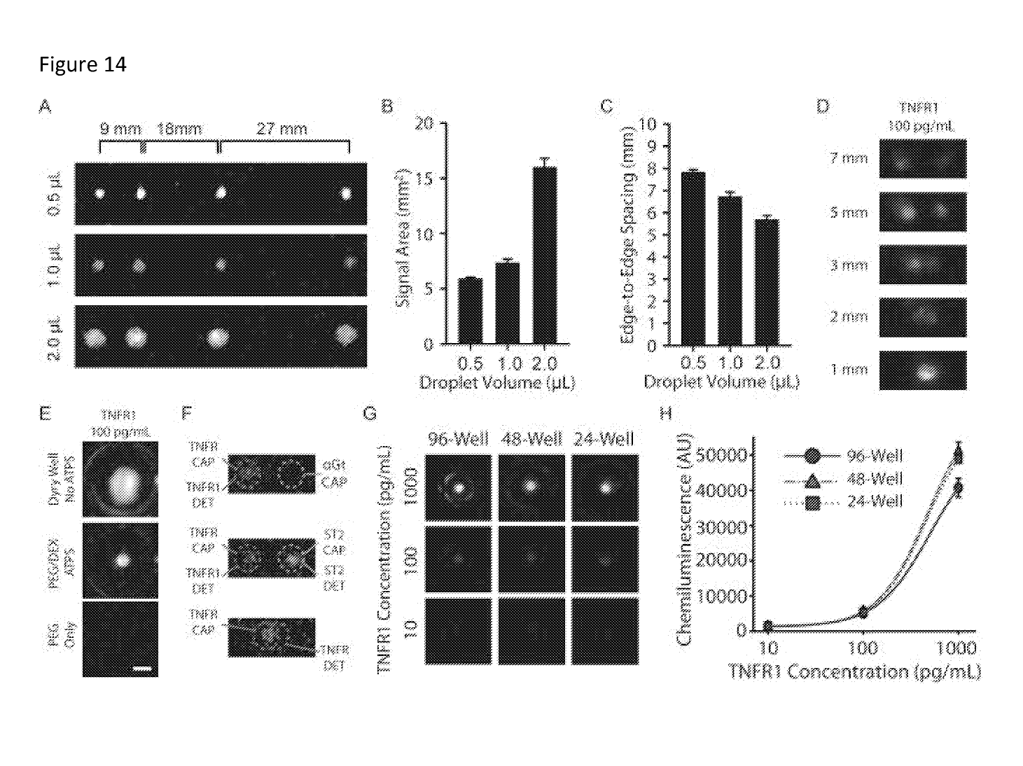

Next the droplet dispensing method was optimized. Droplet spacing could be easily controlled by adjusting the tip spacing on a multipipettor (FIG. 14A). Signal area scaled with volume and reached a minimum of 5.85 mm2, which was limited by the minimum with a dispensedable volume of 0.5 μL due to the viscosity of the polymer solutions (FIG. 2B). As droplet volume increased the edge-to-edge spacing between signal areas decreased, demonstrating that while the assay is compatible with larger droplet sizes, the droplet volume can limit the number of samples that can be multiplexed for a single sample pool (FIG. 14C). It was also demonstrated that when using a handheld micropipettor there was an important droplet spacing of 3 mm before 0.5 μL droplets overlapped and merged (FIG. 14D). To confirm that an ATPS was useful to localize the antibodies, the detection antibody was spotted on a dried well that had been treated with capture antibody and antigen (FIG. 14E). The detection antibody spread across the well producing a large signal area that would preclude multiplexing. Dextran was useful for delivery of the detection antibody in PEG because detection antibody could not come into contact with the plate when dispensed into PEG with a PBS carrier solution (FIG. 14E). Finally, the system was compatible with a variety of polystyrene substrates including large dishes (FIG. 14A) and 96-, 48-, and 24-well plates (FIG. 2G-H).

### Crosstalk Free ATPS-ELISA

An important finding of the optimization was that ATPSs prevented cross talk between detection antibodies. This was confirmed in a three part experiment (FIG. 14F). First, closely spaced droplets of capture antibodies were deposited; one recognizing the antigen of interest (mouse anti-human) and the other (an anti-goat antibody) recognizing the detection antibody (goat anti-human). The goat detection antibody was then deposited above the mouse capture antibody-antigen complex in DEX. Upon developing the plate it was clear that the goat antibody did not escape from the dextran treated region, as even a small amount of antibody that diffused to the anti-goat treated region would bind and produce considerable signal. A duplex experiment was next performed with capture and detection antibodies for TNFR1 and ST2, where both the capture and detection antibodies were again spotted (FIG. 14F). Differential signal intensity between the two spots was observed, again demonstrating that cross talk did not occur. The final demonstration of the spatial localization was partial overlap of the capture and detection antibodies (FIG. 14F). A “cat eye”-shaped signal area was observed, which is only possible if the detection antibody is retained in the dextran droplet, again demonstrating that the system eliminates crosstalk.

### Singleplex and Multiplex Detection of Standards

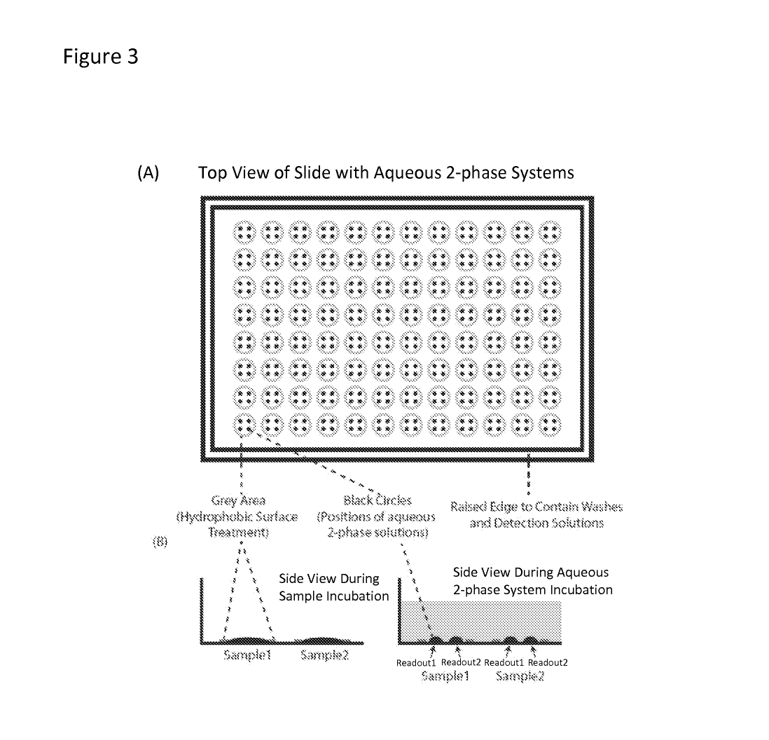

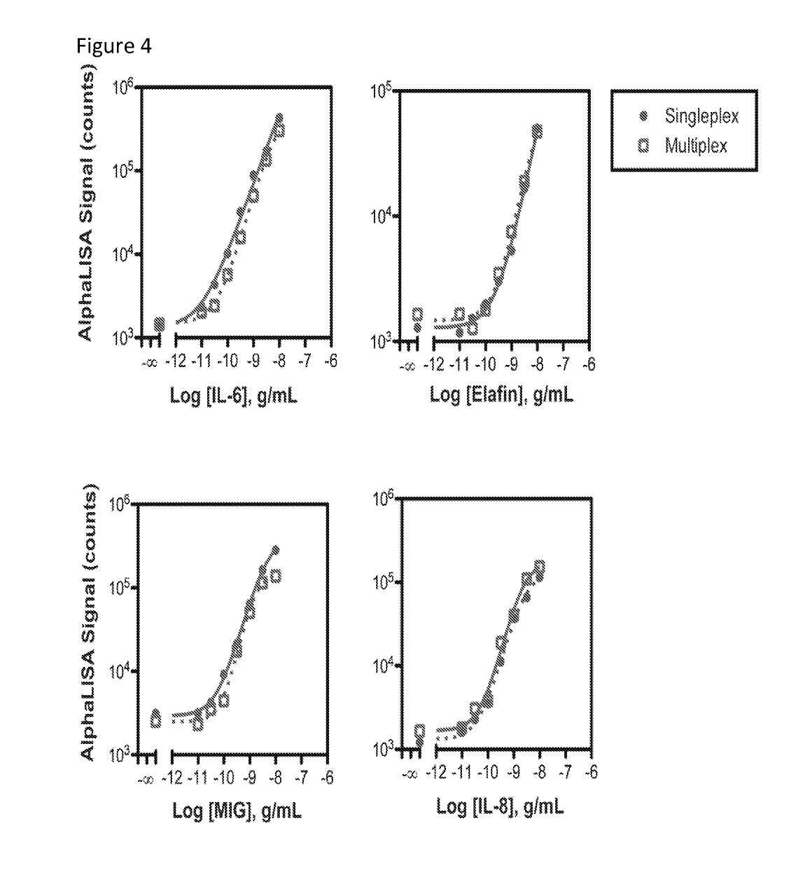

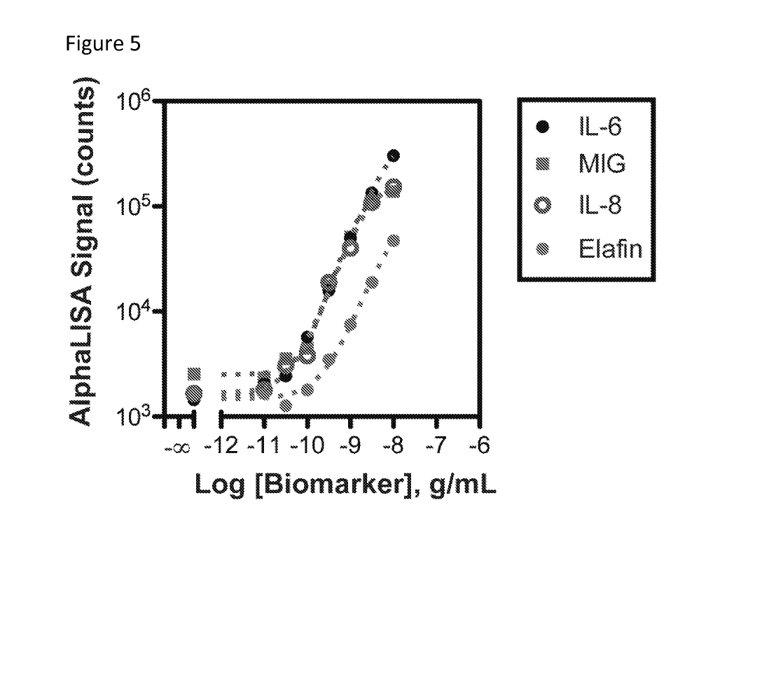

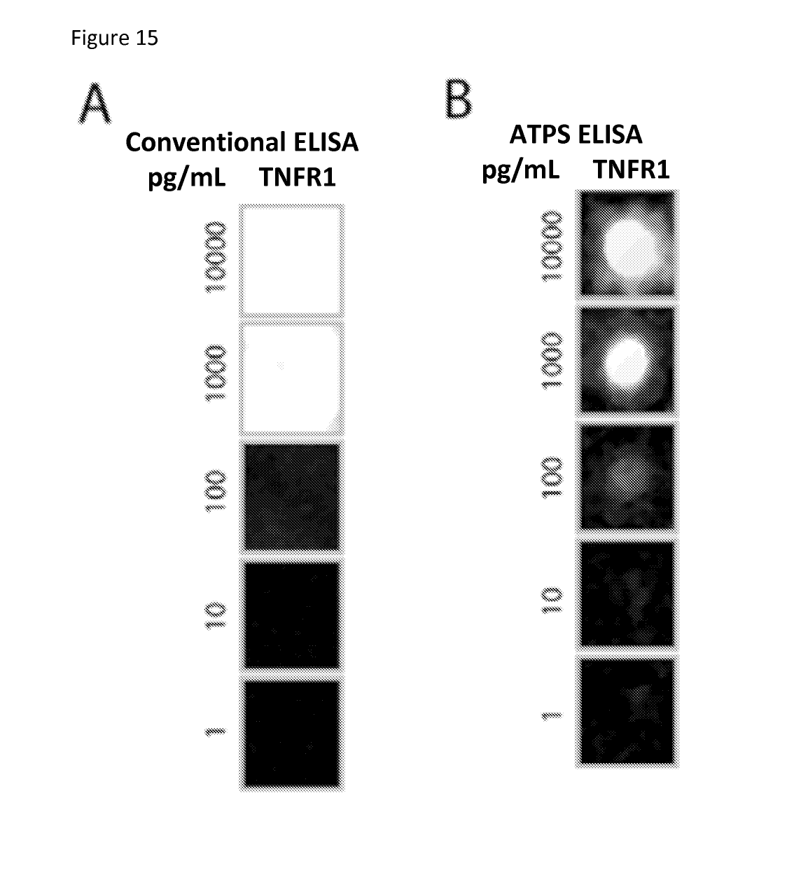

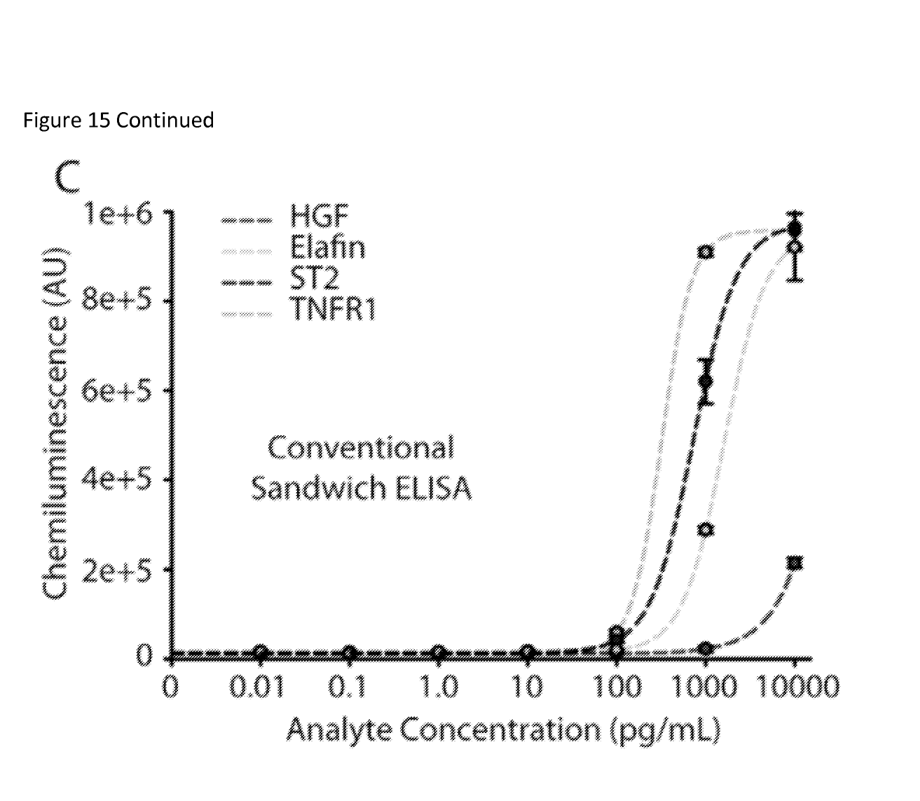

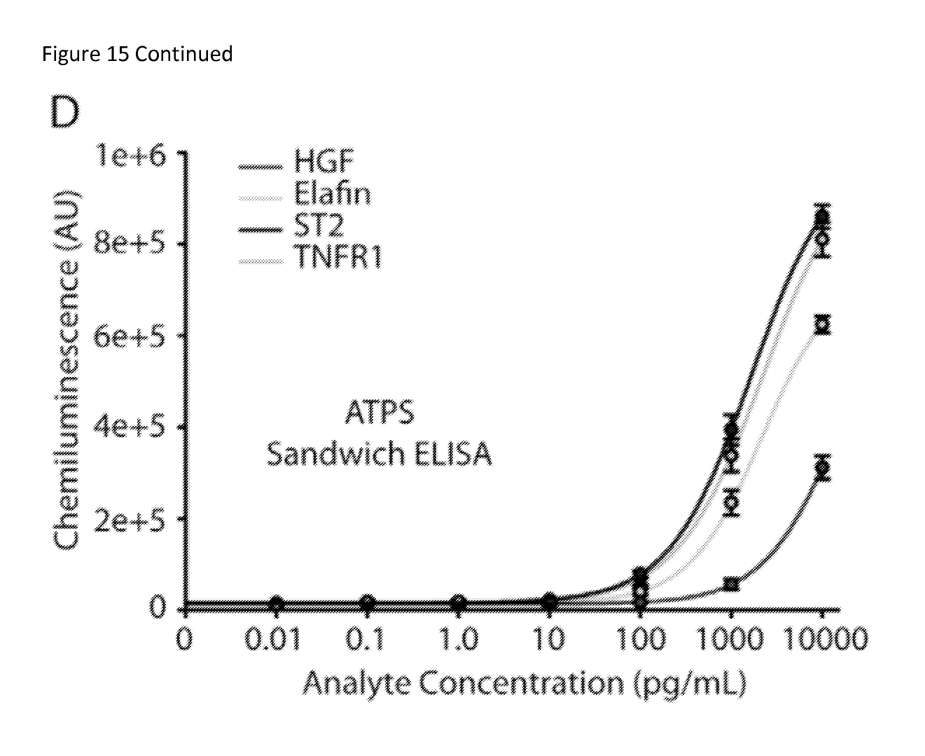

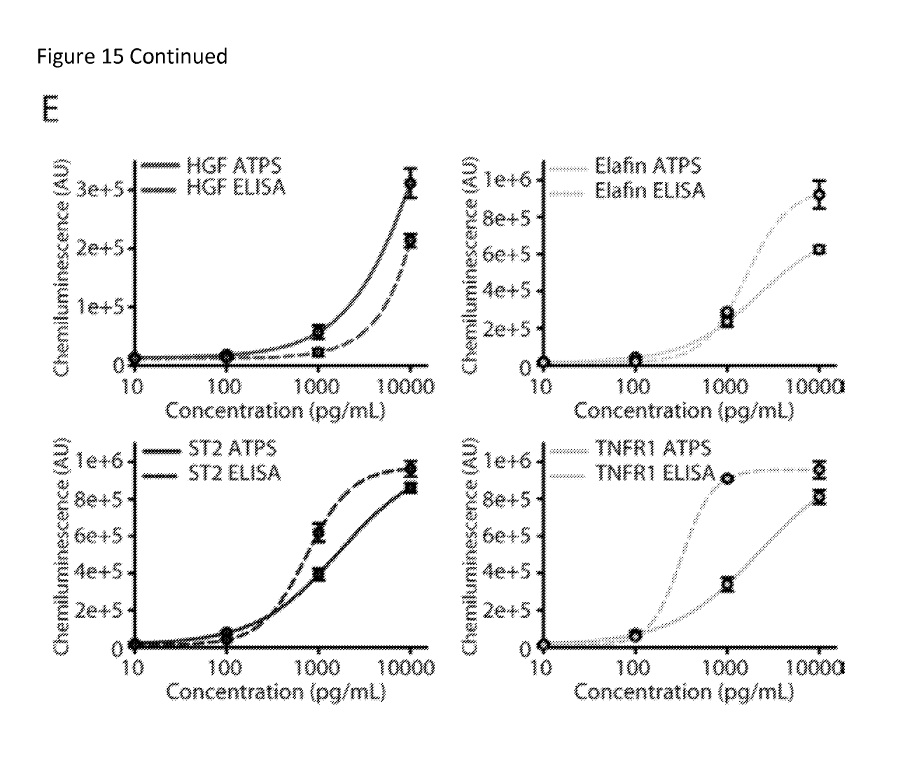

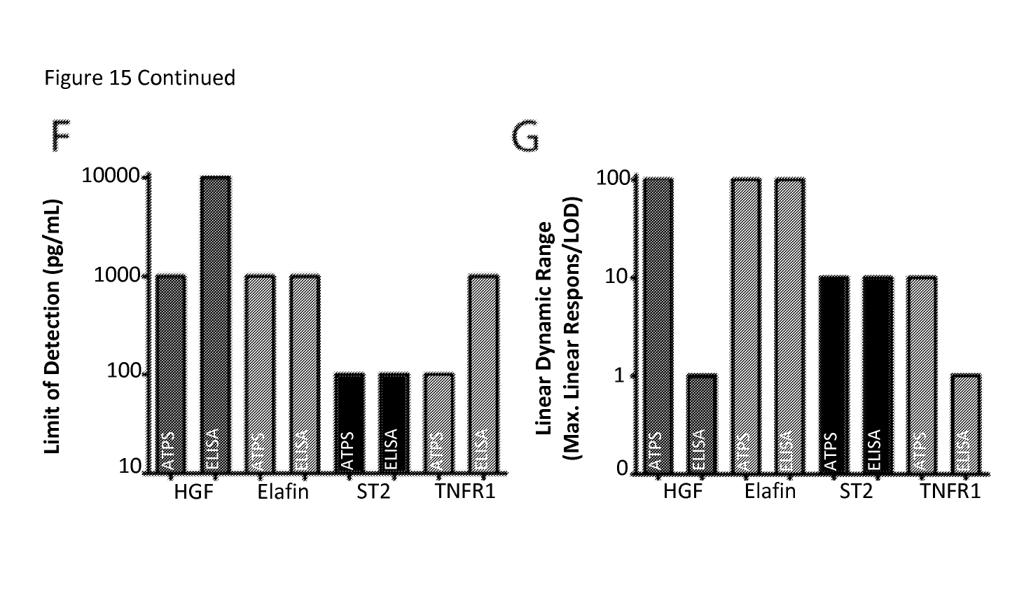

To characterize the performance of the system relative to conventional ELISA standard curves were generated for known antigen concentrations over a range of 5 orders of magnitude (FIG. 3A-D). Four biomarkers for acute GVHD were selected for the initial characterization (HGF, ST2, Elafin and TNFR1). In general the standards curves for ATPS-ELISA were comparable to conventional ELISA (FIG. 15C-E). The limit of detection was lower for the ATPS-ELISA for some biomarkers (HGF and TNFR1). The linear detection range was also broader for HGF and TNFR1. Together these data indicate that it is possible for ATPS-ELISA to outperform conventional ELISA in terms of sensitivity and linear dynamic range.

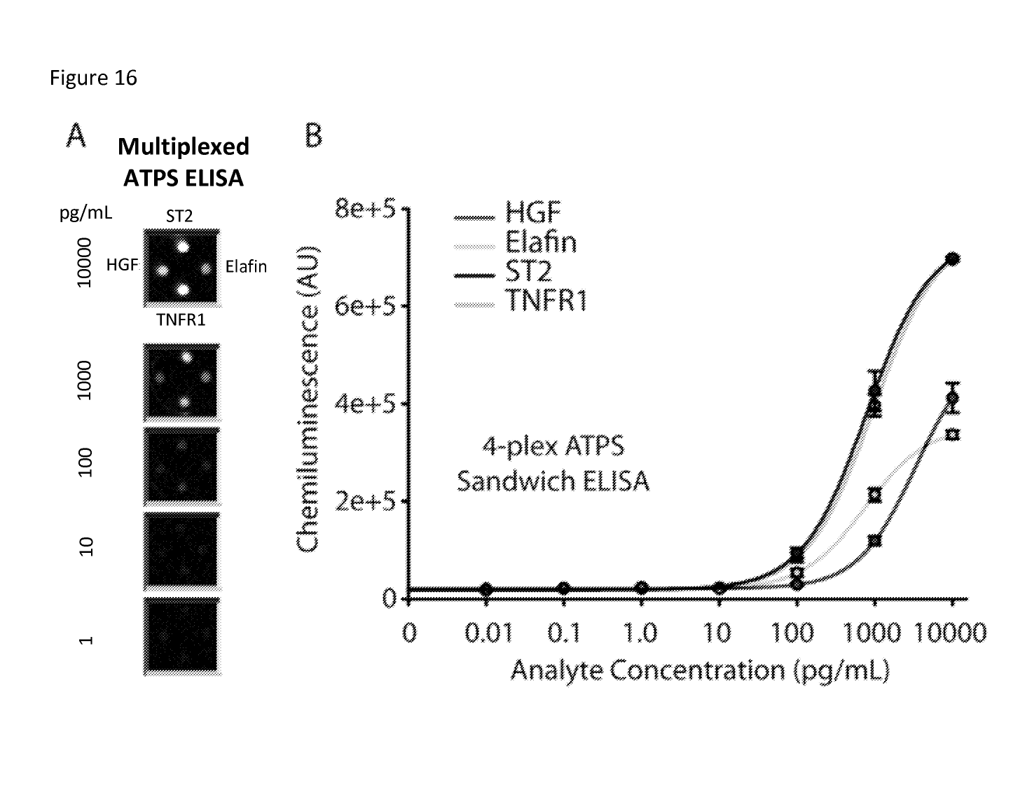

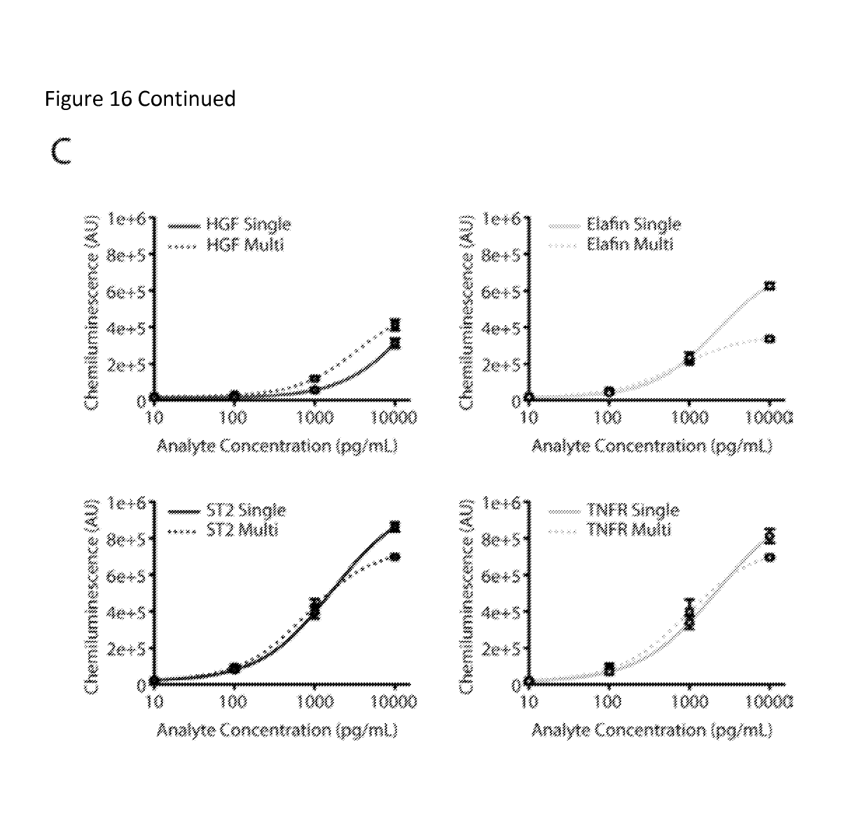

The assay was next performed in multiplex format for the four GVHD biomarkers (FIG. 16A-B). For this experiment the capture antibodies were applied as a cocktail while the detection antibodies were spotted using ATPS. The results we obtained for multiplexing were qualitatively very similar to those observed for singleplex. Upon direct comparison, the curves from independent experiments conducted with singleplex and multiplex formats completely overlapped over the linear detection range (FIG. 16C). These results indicate that ATPS-ELISA can serve as a diagnostic tool for quantitatively detecting acute GVHD biomarkers.

### Screening of GVHD in Plasma Samples

A major challenge for most immunoassays is accommodating patient plasma samples. Plasma obtained from different patients is characteristically different and can vary depending on the health of the patient as well as extraction and handling procedures. This results in high variability in immunoassay compatibility among plasma samples. Furthermore, a phenomenon known as matrix effect is commonly encountered during immunoassay development. A matrix effect occurs when there are differences between the non-antigen components of the standard and the sample (the matrix). Antigen detection may be affected by a difference between the standard diluent and the plasma sample matrix. Finally, when testing multiple biomarkers a complicating factor is the difference in relative abundance of each biomarker.

In the system, matrix effect was accounted for by matching the standard diluent to the sample matrix, so that unknown values could be accurately predicted from the standard curves. It was found that a standard diluent containing 1:10 healthy pooled plasma in PBS with 1% FBS served this purpose well, while it was optimal to dilute the sample itself in a solution of 1% FBS to a final dilution of 1:10. HGF is only a moderately predictive biomarker for GVHD, so for validating our assay on plasma samples we elected to replace HGF with IL-2Rα. For the 4 biomarkers we tested (IL-2Rα, TNFR1, ST2, and Elafin), it was found that the 1:10 dilution was sufficient to allow the 4 markers to fall within the detection range of the assay.

To demonstrate that the assay was capable of detecting differential levels of antigen in plasma samples 2 groups, healthy donors whose plasma contains only low levels of these biomarkers and autologous donors who should possess slightly elevated biomarker levels but do not have GVHD were compared. Significantly higher levels of TNFR1, ST2 and IL-2Rα were measured in autologous patients as compared to healthy donors. Elafin was the only marker for which this trend was not observed; however, this can be explained by the fact that healthy individuals often possess elevated levels of elafin due to allergies, asthma, and other lung conditions. These values were compared with a conventional ELISA assay and it was observed identical trends in the mean concentration of the biomarkers for healthy donors and autologous patients. A pairwise comparison using Pearson product-moment correlation revealed a significant correlation between the two data sets, providing evidence that the ATPS-ELISA system is in accordance with conventional ELISA.

Samples from patients from a GVHD study were next tested. The assay was able to differentially detect levels of critical biomarkers in these samples. Patients with GVHD had higher mean plasma concentrations of ST2, TNFR1 and HGF as compared to patients without GVHD. A pairwise Pearson correlation revealed a significant positive correlation between the ATPS-ELISA data and the conventional ELISA data.

### EXAMPLE 2

### Methods

### Statistical Analysis

All plots and statistical analyses were carried out in Sigmaplot and Sigmastat (Systat Software). Standard curves were fitted using a four parameter logistic function that was used to predict unknowns. The limit of detection (LoD) was determined from the equation LoD=LoB+1.645 (SDlow concentration sample), where SD is the standard deviation and LoB is the limit of blank. LoB was calculated from LoB=mean_{blank}+1.645(SD_{blank}). Linear dynamic range (LDR) was determined using LDR=Maximum linear response/LoD. To compare ATPS-ELISA values with conventional ELISA values a Pearson product-moment correlation test was used with values of |0.3-0.5| and |0.5-1.0| considered to be medium strength and strong correlations respectively.

### AlphaLISA Assay Procedure

Human IL-6 (AL223C), human IL-8 (AL224C), and human CXCL9/MIG (AL280C) AlphaLISA immunoassay kits were purchased from Perkin Elmer and performed according to manufacturer's protocol. The assay buffer contained 25 mM HEPES, 1 mg/mL Dextran (Mw. 500,000), 0.1% casein, 0.5% Triton X-100, and 0.05% Proclin-300. Standards, biotinylated detection antibody, and acceptor beads solutions were prepared in the assay buffer. The assay was performed in 384-well white polypropylene conical-bottom microplates (4307, Thermo Scientific). 2 μL standard or plasma sample was incubated with 8 μL of anti-analyte acceptor beads and biotinylated detection antibody for 1 hour at room temperature. 10 μL streptavidin-coated donor beads were added to the mixture and incubated an additional 30 minutes. The final concentration of reagents per well was 10 ug/mL acceptor beads, 1 nM detection antibody, and 40 ug/mL donor beads. The plate was read on a PHERAstar FS reader (BMG LabTech) whereby the bead complex was excited at 680 nm for 1 second and emission signals were collected at 615 nm for 1 second.

### Singleplex Droplet-Based Homogeneous Immunoassay.

18.0% wt/wt antioxidant-free PEG (Mw. 35,000) (Fluka) and 17.0% wt/wt Dextran (Mw. 10,000) were prepared in AlphaLISA buffer in separate conicals. PEG was mixed with standard or sample solutions in a 4:1 volume ratio and the mixture was vortexed. Using a multipipettor, 10 μL of the PEG-analyte mixture was dispensed into 384-well microplates such that each well contains 2 μL standard or sample and 8 μL PEG. Acceptor beads (25 ug/mL) and biotinylated detection antibody (2.5 nM) were prepared in 17% DEX. Using a multipipettor, 1 μL of the DEX/antibody mixture was dispensed in each well and the mixture was incubated at room temperature for one hour. To prevent evaporation, the plates were sealed with a sealing film. 80 μg/mL donor beads were prepared in 17% DEX. At the end of the one hour incubation, 1 μL of the DEX/donor beads was dispensed into each well and incubated for an additional 60 minutes. Since the antibody reagents remain confined in the DEX droplet, the final concentration of the reagents was 12.5 ug/mL acceptor beads, 1.25 nM detection antibody and 40 ug/mL donor beads. The assays were also read on a PheraSTAR FS Plus after bead excitation for 1 second.

### Coupling of Capture Antibody to Beads

Goat anti-human trappin-2/elafin capture antibodies and biotinylated anti-elafin detection antibodies were obtained from R&D Systems (DY1747). Capture antibody was coupled to the acceptor beads as described by Perkin Elmer. Briefly, 8 μL acceptor beads (20 mg/mL) was added to 8 μL PBS in low retention silicon-coated eppendorf tubes and centrifuged for 15 minutes at 4000 rcf. After the supernatant was discarded, 16 ug capture antibody was added to the bead pellet and incubated with sodium cyanoborohydride (15.3 mM) for 24 hours at 37° C. with mild agitation (12 rpm) on a rotary shaker. After 24 hours, unreactive sites on the bead surface are blocked upon addition of carboxymethoxylamine in 800 mM sodium hydroxide for 1 hour at 37° C. and 12 rpm. The bead mixture was centrifuged four times at 4000 rcf for 15 minutes. After each centrifugation step, the supernatant was discarded and the bead pellet resuspended in 100 mM Tris HCl, pH 8.0. After the final centrifugation, the pellet was resuspended in PBS, containing 0.05% Proclin-300 and stored at 4° C.

### Partitioning of Biotinylated Detection Antibodies

Partitioning of the biotinylated detection antibodies was determined by direct enzyme-linked immunosorbent assay (ELISA). 8-10 nM biotinylated detection antibody against each biomarker was added to eppendorf tubes, containing 7% PEG and 7.5% DEX. Contents of each eppendorf tube were well mixed by pipetting and then tubes were centrifuged at 400 rcf for 10 minutes. After centrifugation, the PEG and DEX systems phase separated. 0.3 μL of the PEG phase, interface, and DEX phase was sampled for the biomarker tubes as well as a blank sample (no antibody added). Sampled volumes of the phases was dispensed onto square polystyrene Petri dishes and stored overnight at 4° C. Detection antibodies in the sample phases adsorb to the polystyrene dishes. After overnight incubation, the dishes were washed 5 times with wash buffer (PBS, 0.05% Tween-20). Dishes were blocked with 3.0% casein for 1 hour, followed by 5 washes in the wash buffer. Streptavidin-horse radish peroxidase (streptavidin HRP) was added to the plates and incubated for 1 hour at room temperature. During this incubation step, the streptavidin HRP binds to the adsorbed biotinylated detection antibodies. The plates were again washed 5 times and incubated with SuperSignal ELISA Femto Maximum Sensitivity Substrate (37074, Thermo Scientific). Chemiluminescent signal was detected using the FluorChem M (Protein Simple) imaging system.

### Custom Multiplex Plate Design and Fabrication

Multiplex homogeneous bead-based assays were performed in custom-built multiplex plates. Custom plates were fabricated using a proprietary rigid, white opaque material (VeroWhite) in an Objet printer. In this design, the reflective coating of the rigid plastic material allows the emission signal from immunobead complexes to be detected by photomultiplier tubes (PMTs) in the reader. PEG wells occupied an area of 8 mm×8 mm and had a height of 11.4 mm. DEX droplets can easily slide across a flat surface if the surface is tilted. To keep the DEX droplets in discrete locations, DEX wells were designed as flat-bottom conical wells (2.5 mm×2.5 mm). To ensure the custom plate satisfies the Society of Biomolecular Screening specifications for microplates, the DEX wells were arranged in standard 384-well format (24 columns, 16 rows, 4.5 mm spacing). To minimize optical crosstalk between emission signals in neighboring DEX wells, DEX wells were designed with 2.5 mm height and the multiplex plate was read using a 1536 aperture in the PHERAstar FS reader. For multiplexed assays, 18% w/w PEG was mixed in a 4:1 volume:volume ratio with the standard dilutions or plasma sample. The PEG/analyte mixture was vortexed briefly and 100 μL of the PEG/analyte mixture dispensed into each PEG well. Acceptor beads and biotinylated detection antibodies against 4 different antigens (elafin, IL6, IL8, CRP, TNFα or MIG) were dispensed into each of the four DEX wells per larger PEG well.

### Plasma Samples

For measuring biomarker concentrations in patient plasma, plasma was diluted 1:8 in a custom buffer adjusted to pH 7.4, containing 25 mM HEPES, 50 mM sodium chloride, 10 mM sodium ethylenediaminetetraacetic acid (EDTA), 2 mg/mL DEX T500, 0.5% casein, 0.1% bovine gamma globulins, 0.2 mg/mL heterophilic blocking reagent 1 (HBR1) (Scantibodies Laboratories Inc.), 0.1% Tween 20. 2 μL diluted plasma samples were added to 8 μL 18% w/w PEG phase, which was prepared in 25 mM HEPES buffer, 0.1% casein. The DEX phase was prepared in the aforementioned custom buffer and contained the acceptor beads, donor beads, and biotinylated antibodies.

### Results

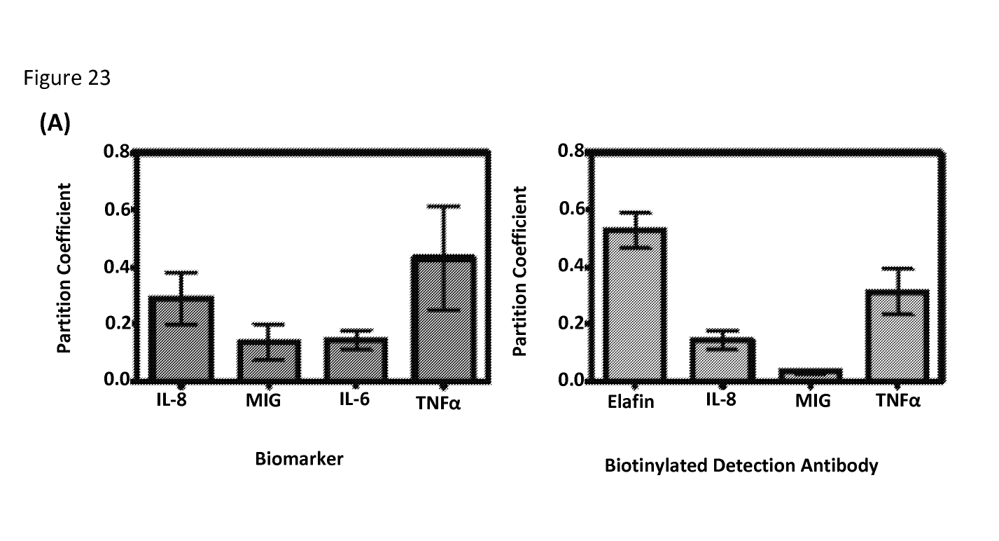

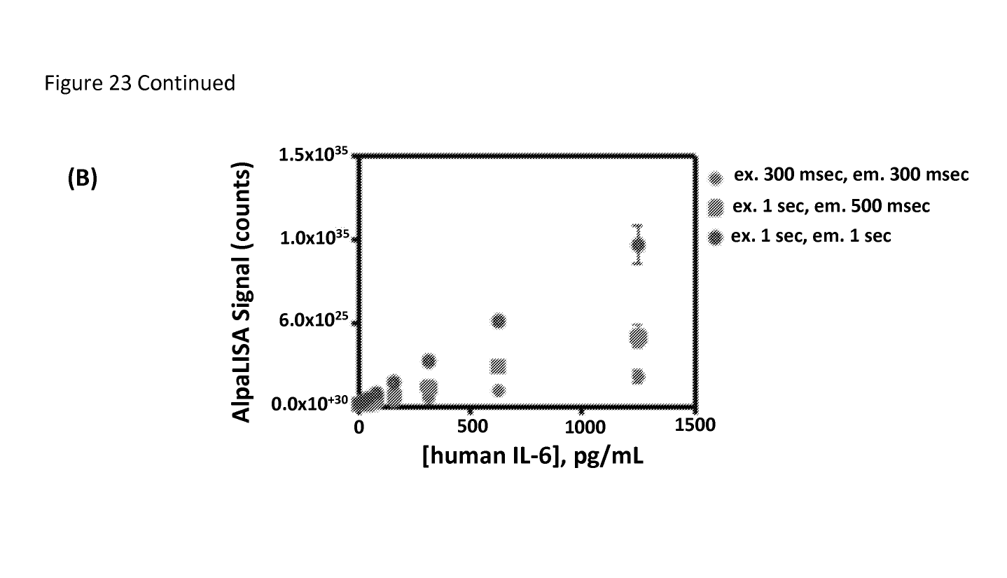

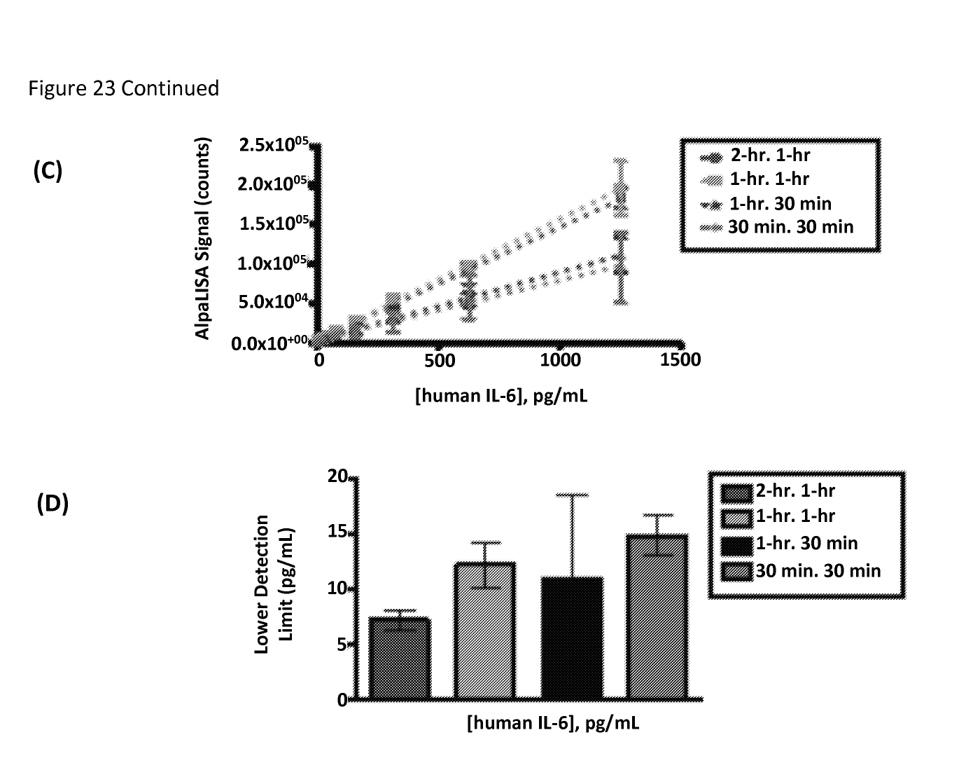

An aqueous two-phase system (ATPS) is composed of two immiscible water-soluble polymers. Because each of the two resulting phases consists of >90% (w/w) water, an ATPS is advantageous for the gentle separation of biological reagents, including proteins (Albertsson 1986). The ability to selectively partition proteins within a completely hydrated environment led to the use of ATPS technology for the partitioning of AlphaLISA bead-based assay reagents. The chosen ATPS-forming polymers are poly(ethylene glycol) (PEG, Mw. 35,000) and Dextran (DEX, Mw. 10,000), where PEG predominantly occupies the upper phase while DEX predominantly occupies the lower phase. The partition coefficient is defined as the ratio between the concentration of the reagent in the PEG phase versus the reagent concentration in the DEX phase. All assay reagents (acceptor beads, donor beads, biotinylated detection antibodies, and antigens) partition stably to the DEX phase of this ATPS (FIG. 23).

Diffusion of antigens into the DEX phase depends upon the partition coefficient of the antigen, the antigen's molecular weight and the viscosity of the ATPS polymers. Of the tested graft-versus-host disease biomarkers, IL-6 was selected to optimize the bead-based assay because (1) it partitions most stably to the DEX phase, (2) it has a molecular weight of 22 kDa, which is considerably larger than elafin (8 kDa) and comparable to TNFα (25.6 kDa). Larger proteins have longer diffusion times. To determine the optimum incubation time required for the miniaturized ATPS bead assays, singleplex IL-6 ATPS assays were performed. Two hour, 1-hour, and 30 minute incubations of antigens in PEG with the 1 μL DEX droplets that contained acceptor beads and detection antibody were compared. After the first incubation time, the second microliter DEX droplet, containing streptavidin donor beads was added for an additional hour or 30 minutes. The results indicate that 30-minute incubations do not provide adequate time for the reaction to go to completion. Instead 1-hour incubations of antigens with the bead-based reagents in DEX provide sufficient time for the assay (FIG. 23). It was also determined that exciting the acceptor beads for 1 second at 680 nm, followed by emission signal collection for 1 second at 615 nm gives improved signal-to-noise ratios compared to 300 millisecond excitation and emission times. Improved signal-to-noise ratios allow more accurate quantification of unknown samples.

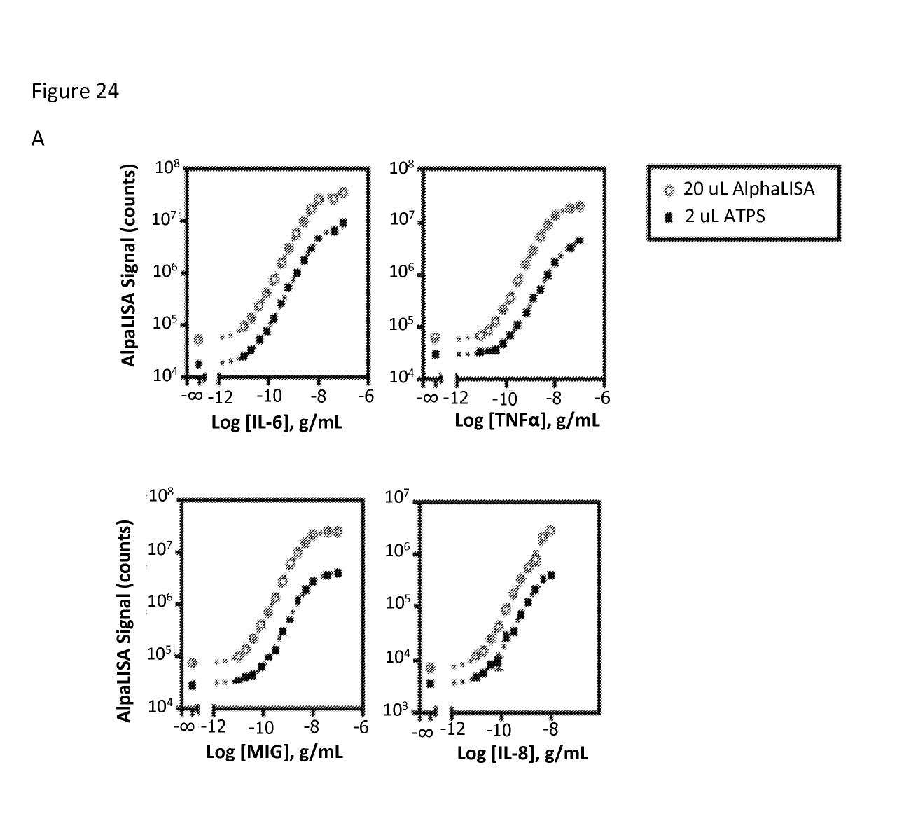

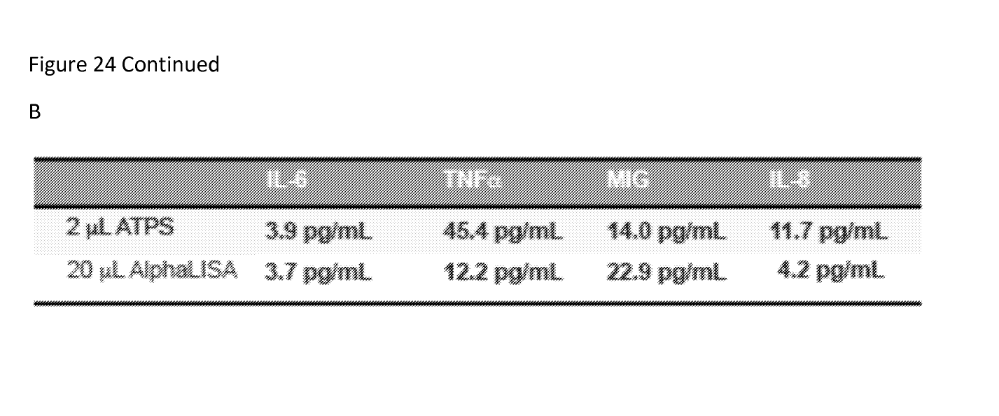

A large linear range is helpful for biomarker assays since biomarkers are present at various concentrations in the blood. The 2-microliter ATPS assays were compared to conventional 20 uL AlphaLISA assays for five biomarkers (IL-8, IL-6, MIG, elafin and TNFα). For all assays, the similar dynamic range of 5 to 6 logs to the conventional AlphaLISA assays was obtained (FIG. 24). Lower detection limits (LDLs) of the assays was calculated as background signal+3× standard deviation of the blank. Based on these calculations, similar picomolar detection limits to conventional AlphaLISA were obtained (3.9 pg/mL IL-6, 45.4 pg/mL TNFα, 14.0 pg/mL MIG, 11.7 pg/mL IL-8, and 300 pg/mL elafin). It is contemplated that the detection limit for TNFα is greater than three of the measured biomarkers because TNFα had the greatest partition coefficient in the chosen ATPS system.

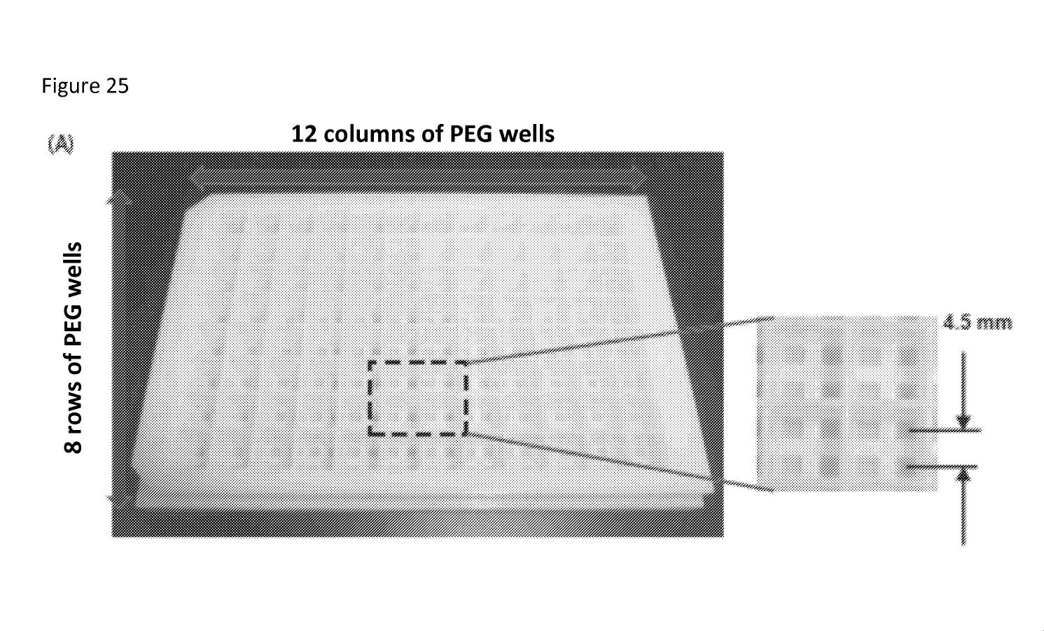

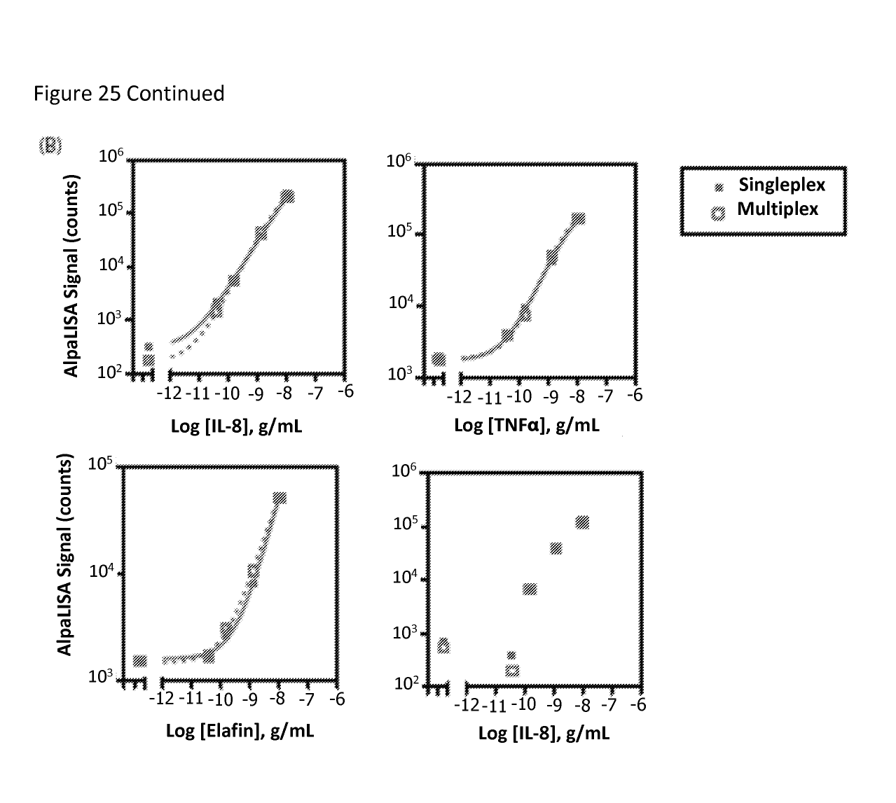

Using the custom-designed white, opaque, multiplex microplates, multiplex GVHD biomarker assays were performed without any antibody crosstalk using homogeneous assay bead reagents. It is important to note that multiplexing AlphaLISA is impossible in its current format. Singleplex calibration curves of IL-8, IL-6, Elafin, and MIG overlap with multiplex calibration curves, indicating the absence of detection antibody crosstalk as well as the absence of optical crosstalk (FIG. 25). Because detection antibodies and acceptor beads for all markers remain confined within each 2-microliter assay droplet, detection antibodies cannot bind nonspecifically to the incorrect antigens. Optical crosstalk between neighboring assay droplets was considered due to: (1) bleaching caused by excitation of adjacent assay droplets, (2) glow from an adjacent droplet at the time of measurement, and (3) afterglow from an adjacent droplet a few milliseconds after measurement. To prevent optical crosstalk, the walls of the DEX well were designed to be 2.5 mm in height. This height is sufficient to prevent strong chemiluminescent signal in one assay droplet from masking weaker signals in neighboring droplets. To further prevent optical crosstalk, the PEG/sample wells were used in a staggered configuration.

We claim:

1. An assay system, comprising:
a) a solid support comprising at least one first defined area; and
b) a first solution comprising a first polymer or solute and reagents for detecting the presence or absence of one or more analytes in a test sample; and
c) a second solution comprising a second polymer or solute and said test sample, wherein said second solution has a different density than said first solution, and wherein said first and second solutions form an aqueous two-phase system when mixed.

2. The system of claim 1, wherein said defined area is defined by a chemical or topographical barrier.

3. The system of claim 1, wherein said first defined area comprises two or more second defined areas within said first defined area.

4. The system of claim 2, wherein said first and second topographically defined areas are selected from the group consisting of wells, hydrophobic lines, and physical barriers.

5. The system of claim 1, wherein said solid support is selected from the group consisting of a plate, a multiwall plate, and a slide.

6. The system of claim 1, wherein said solid support is constructed from a material selected from the group consisting of plastic, paper, hydrogel, tape, cloth, yarn, glass, conducting material, and thread.

7. The system of claim 1, wherein reagents comprise an antibody specific for said analyte.

8. The system of claim 1, wherein said test sample is selected from the group consisting of plasma, serum, urine, saliva, body fluids, cell culture fluid, and cells.

9. The system of claim 1, wherein said solid support is a 6, 24, 48, 96, 384, 1536, 3456 or 9600 well microplate.

10. The system of claim 1, wherein said first polymer or solute is selected from the group consisting of polyethylene glycol (PEG), dextran (DEX), DEX-methylcellulose, DEX-polyvinyl alcohol, PEG-DEX sulfate, polyvinyl alcohol-DEX sulfate, hydroxypropyldextran-DEX, DEX sulfate-methylcellulose, and ethylene oxide-propylene oxide (EOPO).

11. The system of claim 1, wherein said second polymer or solute is selected from the group consisting of polyethylene glycol (PEG), dextran (DEX), DEX-methylcellulose, DEX-polyvinyl alcohol, PEG-DEX sulfate, polyvinyl alcohol-DEX sulfate, hydroxypropyldextran-DEX, and DEX sulfate-methylcellulose, and ethylene oxide-propylene oxide (EOPO).

12. The system of claim 1, wherein said first or second solutions comprise two or more polymers.

13. The system of claim 1, wherein said reagents comprise one or more of primary and secondary antibodies that specifically binds to analytes in said test sample.

14. The system of claim 13, wherein said antibodies are attached to the surface of said solid support.

15. The system of claim 14, wherein one or more of said first solution and said second solution is dehydrated.

16. The system of claim 14, wherein said second solution comprises one or more reagent selected from the group consisting of primary antibodies, secondary antibodies, antibody-conjugated acceptor beads, biotinylated detection antibodies, probes, enzymes, amplification reagents, and donor beads.

17. The system of claim 1, wherein said solid support is fabricated from a thermoplastic polymer.

18. The system of claim 1, wherein a surface of said solid support is hydrophobic, hydrophilic or omniphobic.

19. The system of claim 1, further comprising a detection system selected from the group consisting of colorimetric, fluorescent, fluorescence polarization or lifetime readings, refractive index change, and electrochemical detection systems.

20. A method, comprising:
a) contacting a solid support comprising at least one first defined area; and two or more second defined areas within said first defined area; with: i) a first solution comprising a first polymer or solute and reagents for detecting the presence or absence of at least one analyte in a test sample; and ii) a second solution comprising a second polymer or solute and said test sample, wherein said second solution has a different density than said first solution, and wherein said first and second solutions form an aqueous two-phase system when mixed; and
b) detecting the presence of said analyte in said sample.
